(19) United States
(12) Patent Application Publication
(10) Pub. No.: US 2017/0340688 A1
(43) Pub. Date: Nov. 30, 2017

(54) TITRATED EXTRACTS OF CYNARA SCOLYMUS AND USES THEREOF

(71) Applicant: ABOCA S.P.A SOCIETÀ AGRICOLA, Sansepolcro (IT)

(72) Inventor: Valentino MERCATI, Sansepolcro (IT)

(21) Appl. No.: 15/528,846

(22) PCT Filed: Nov. 24, 2015
(86) PCT No.: PCT/IB2015/059073

§ 371 Date: May 23, 2017

(30) Foreign Application Priority Data
Nov. 25, 2014 (IT) 102014902312096

(51) Int. Cl. A61K 36/28; A61K 31/192; A61K 31/216; A61K 31/343

(52) U.S. Cl. CPC A61K 36/28; A61K 31/343; A61K 31/192; A61K 31/216; A61K 2236/00

(57) ABSTRACT

The present invention relates to a titrated extract of Cynara scolymus, to titrated fractions of extract of Cynara scolymus or titrated mixtures of said extract with one or more of said titrated fractions or mixtures of said fractions, and to compositions and kits comprising them, for the prevention and/or the treatment of a pathological condition characterised by a constitutive activation of the STAT3 transcription factor,

### TECHNICAL FIELD OF THE INVENTION

The present invention relates to a titrated extract of Cynara scolymus, to titrated fractions of extract of Cynara scolymus or titrated mixtures of said extract with one or more of said titrated fractions or mixtures of said fractions in combination with one or more chemotherapeutic or anti-inflammatory drugs, and to compositions and kits comprising them, for the prevention and/or the treatment of a pathological condition characterised by a constitutive activation of the STAT3 transcription factor.

### PRIOR ART

In recent decades, much evidence in literature indicates the fundamental role of transcription factors belonging to the STAT family in a wide variety of pathologies, such as in inflammatory pathologies that promote tumours, and in tumours themselves. STAT proteins are cytoplasmic transcription factors of which the phosphorylation/activation (on specific residues of serine and/or tyrosine due to the action of the families of JAK, or Janus kinase proteins) determines the dimerization of two STAT monomers, the translocation of the dimer in the nucleus, the binding to elements of the DNA of STAT-specific target genes, and the induction of gene transcription. The family of the STAT factors consists of seven members (coded by the genes STAT1, STAT2, STAT3, STAT4, STAT5A, STAT5B and STAT6) with various biological functions that include roles in differentiation, proliferation, development, apoptosis and cell inflammation. One characteristic of the proteins coded by these genes is that of having a dual role, more specifically a role of transduction of the signal in the cytoplasm and of transcription factor in the nucleus. In particular, a constitutive activation of STAT3 and to a lesser extent of STAT5 has been associated in various neoplasias with the deregulation of some intracellular pathways, including those involved in the survival of the tumour and in the proliferation of the tumour cell, but also in the process of angiogenesis and metastasis of the tumour itself.

Yu H. et al in a review published on Nature in 2009 (Nature Reviews Cancer 9, 798-809: 2009) reported that the persistent activation of STAT3 induces inflammation that promotes the cancer and regulates genes crucial for the inflammation and the tumour microenvironment. Genes activated by STAT3 are shown in Tables 1 and 2 of the above-mentioned work, and some inhibitors of the activation of STAT3 are also described among natural substances, such as curcubitacin, resveratrol, galiellalactone and indirubin, however it is stated that the mechanisms of actions by which these substances act are unknown.

In any case, the work states that the modulation of STAT3 is a new, more effective and highly advantageous approach for treating cancer, reporting that the ablation of the STAT3 gene in various tumour models led to inhibition of tumour growth.

The constitutive activation of STAT3 has been reported in a large number of tumours, including breast cancer, prostate cancer, squamous-cell carcinoma of the head and neck, multiple myeloma, lymphoma and leukaemia, brain tumours, colon cancer, Ewing's sarcoma, stomach cancer, oesophageal cancer, ovarian cancer, nasopharyngeal cancer, and pancreatic cancer (Table 1 below). For many types of cancer, high levels of activated STAT3 have been linked to a poor prognosis. The activation of STAT3 blocks apoptosis and increases cell proliferation and cell survival, promoting angiogenesis and metastasis and inhibiting the anti-tumour immune responses. Tumour cell lines in which STAT3 is constitutively activated require the continuous activation of STAT3, a phenotype that has been defined as “dependence on oncogenes” (Johnston P A and Grandis R G, Mollnterv; 11 (1); 18-26:2011).

Malignant plural mesothelioma (MPM) is an aggressive tumour derived from the mesothelial cells of the chest cavities, and, although chemotherapy (often if pemetrexed is used) improves the survival time in patients with non-operable MPM, the average global survival time is just 12 months. It has been reported recently that a potential molecular therapeutic target for MPM is the interleukine-6 signalling pathway (IL-6)/JAK/STAT3 activated by the high level of IL-6 present in pleural liquid of patients with MPM. The bind of IL-6 to its receptor causes a conformational change in the receptor that initiates JAK activation, which in turn initiates the dimerization of the STAT3 transcription factor, and the STAT3 dimer translocates in the nucleus, thus determining the initiation of the transactivation of various target genes.

This pathway is key for the occurrence of haematopoiesis, of the immune response and of oncogenesis. In addition, it has also been demonstrated that the dysfunction of the JAK/STAT3 system is involved in the development of cancer.

In addition, a broad description of the role played by STAT3 in the development and in the progression of the tumour is ever present in the literature. A constitutive activation of STAT3 has been observed both in blood tumours (multiple myeloma, leukaemia, lymphoma) and in solid tumours (melanomas, carcinoma of the ovaries, of the prostate and of the renal cells, pancreatic adenocarcinoma, lung cancer, breast cancer and brain cancer). For greater depth, Table 3 below, taken from Turkson J and Jove R, Oncogene; 19(56); 6613-26: 2000, indicates numerous tumours directly associated with the anomalous activation of STAT3. In particular, this anomalous activation seems to be caused by the action of transforming tyrosine kinases, such as v-Src, v-Ros, v-Fps, Etk/BMX and Lck, or by an anomalous signal induced by the autocrine or paracrine release of cytokines. The constitutive activation of STAT3 leads to a greater expression of genes coding for inhibitors of apoptosis (for example Bcl-xL, Mcl-1), regulators of the cell cycle (for example cyclin D1/D2, c Myc) and inducers of angiogenesis (for example VEGF: Vascular Endothelial Growth Factor). Lastly, it has been demonstrated recently that apart from having a key role in tumourigenesis, the constitutive activation of this transcription factor confers resistance to the death induced by chemotherapeutic agents (Aggarwal B. B. et al. Ann. N.Y. Acad. Sci. 1091; 151-69: 2006).

A variety of clinical research has demonstrated that, in vivo, solid tumours grow and develop in an environment with low levels of 02 that make the tumour itself insensitive to the signals of cell death and resistant to radiotherapy and chemotherapy treatments; on the other hand, the hypoxia promotes angiogenesis, proliferation and metastatic ability. The aggressiveness of the tumour in this context seems to be associated with the activation and stabilisation of the factor of HIF-1α both by the hypoxia and by the hyperactivation of STAT3.

For this reason, an anti-cancer therapy based on the targeting of the factor STAT3 is highly desirable (Niu G. et al. Mol Cancer Res, 6 (7); 1099-105: 2008). Table 1, shown below, is taken from the work of Aggarwal B. B. et al. 2006 and shows a list of tumours that express constitutively active STAT3, activators of STAT3, genes regulated by STAT3 and inhibitors of STAT3

TABLE 1

| Constitutive STAT3 | Activators | Genes | Kinases | Inhibitors |
|---|---|---|---|---|
| Haematopoietic tumours | EGF | Antiapoptosis | Non-receptor | Synthetic |
| Multiple myeloma | IL-6 | Bcl-x_{L} | tyrosine kinases | AG490 |
| HTLV-1-dependent | IL-5 | Bcl-2 | JAK | Sodium salicylate |
| leukaemia | IL-9 | Mcl-1 | JAK2 | Atiprimod |
| CLL | IL-10 | cIAP-2 | JAK3 | BMS-354825 |
| CML | IL-12 | Survivin | TYK2 | Ethanol |
| AML | IL-22 | Cell cycle | Src | Nelfinavir |
| Large granular lymphocyte | TNF-α | progression | Receptor tyrosine | PS-341 |
| leukaemia | MCP-1 | Cyclin D1 | kinases | R115777 |
| Erythroleukaemia | GCSF | c-Myc | EGFR | WP-1034 |
| Polycythaemia vera | GMCSF | c-Fos | ErbB-2 | Platinum compounds |
| EBV-related/Burkitt's | CSF | p21 | Gp130 | 15-Deoxy-delta |
| Mycosis fungoides | LI F | Tumour invasion and | Grb2 | 12,14-PGJ2 |
| Cutaneous T cell lymphoma | OSM | metastasis | Serine kinases | UCN-01 |
| HSV saimiri-dependent (T | IFN-γ | MMP-2 | JNK | Statin |
| cell) | MIP-1α | MMP-9 | P38MAPK | Peptides |
| Hodgkin's disease | RANTES | β-catenin | ERK | SOCS3 |
| Anaplastic lymphoma | SLF | VEGF | Tyrosine | PIAS |
| Solid tumours | UVB | hTERT | phosphatase | GRIM-19 |
| Breast cancer | Osmotic shock | IRF-1 | SHP2 | Adiponectin |
| Brain tumour | Progestin | NLK | | Duplin |
| Colon carcinoma | LPS | MyD88 | | SSI-1 |
| Ewing's sarcoma | Tobacco | RANKL | | α-Thrombin |
| Gastric carcinoma | HCV | TNF | | Lipoxin A4 |
| Lung cancer | | β-macroglobulin | | DIF-1 |
| Nasopharyngeal cancer | | SOCS | | PTPεC |
| Ovarian carcinoma | | Angiotensinogen | | STAT3-DN |
| Pancreatic adenocarcinoma | | Antichymotrypsin | | Decoy peptide |
| Prostate carcinoma | | | | Naturals |
| Renal cell carcinoma | | | | |
| SCCHN cancer | | | | Flavopiridol |
| | | | | Indirubin |
| | | | | Magnolol |
| | | | | Resveratrol |
| | | | | Piceatannol |
| | | | | Parthenolide |
| | | | | EGCG |
| | | | | Curcumin |
| | | | | Cucurbitacin |
| | | | | Others |
| | | | | Rituximab |
| | | | | GQ-ODN |
| | | | | Retinoic acid |
| | | | | STA-21 |
| | | | | EKB569 |

Key:
STAT, signal-transducer-and-activator-of-transcription; CLL, chronic lymphocytic leukaemia; CML, chronic myeloid leukaemia; AML, acute myelogenous leukaemia; SCCHN, squamous cell carcinoma of the head and neck, HTLV, human T cell lymphotropic virus; EBV, Epstein-Barr virus; Nelfinavir, HIV-1 protease inhibitor; R115777, farnesyl transferase inhibitor; AG490 and piceatannol, tyrosine kinase inhibitors; PIAS, protein inhibitor of activated STAT3; GQ-ODN, G-quartet oligonucleotides; SOCS, suppressor of cytokine signalling; GRIM, gene associated with retinoid-IFN-induced mortality; EGCG, epigallocatechin-3-gallate; SSI, STAT-induced STAT inhibitor; PTPεC, protein tyrosine phosphatase εC; DN, dominant negative; EKb-569, EGF-R inhibitor; DIF-1, differentiation-inducing factor-1; JAB, SH2-domain-containing protein; IL, interleukin; TNF, tumour necrosis factor; MDA, melanoma differentiation antigen; MCP, monocyte chemoattractant protein; GCSF, granulocyte colony-stimulating factor; LIF, leukaemia inhibitory factor; OSM, oncostatin M; IFN, interferon; MIP, macrophage inflammatory protein; RANTES, regulated upon activation, normal T cell expressed and secreted; EGF, epidermal growth factor; LPS, lipopolysaccharide; VEGF, vascular endothelial growth factor; MMP, matrix metalloproteinase; hTERT, human telomerase reverse; SLF, steel factor, HCV, hepatitis C virus

Table 2 is taken from Johnston P A and Grandis R G 2011 and correlates STAT3 with numerous tumours, confirming the fact that STAT3 is effectively a target of interest for anti-cancer therapies.

TABLE 2

| Characterisation of | Inauspicious | Abnormality | Models of |
|---|---|---|---|
| tumours with | prognosis correlated | upstream and | xenotransplantion |
| increased expression | with high levels of | downstream of the | responsive to the |
| of STAT3 and activity | STAT3 | signal of STAT3 | inhibition of STAT3 |
| Leukaemia | Renal cell carcinoma | Elevated expression | Squamous-cell |
| Lymphoma | Colorectal cancer | of EGFR | carcinoma of the head |
| Multiple myeloma | Ovarian carcinoma | Constitutively | and neck |
| Breast cancer | Gastric carcinoma | activated EGFR-RTK | Glioblastoma |
| Prostate carcinoma | Intestinal-type gastric | Overexpression of | Myeloproliferative |
| Lung cancer | adenocarcinoma | SFK | neoplasms |
| Lung cancer (not small | Squamous-cell | Hyperactivated JAK | Carcinoma of the renal |
| cell) | carcinoma of the | Elevated levels of | cells |
| Carcinoma of the renal | cervix | TNF-α/IL-6 | Breast cancer |
| cells | Osteosarcoma | | Lung adenocarcinoma |
| Hepatocellular | Epithelial carcinoma | | Acute lymphoblastic |
| carcinoma | of the ovary | | leukaemia |
| Cholangiocarcinoma | | | |
| Ovarian carcinoma | | | |
| Pancreatic | | | |
| adenocarcinoma | | | |
| Melanoma | | | |
| Squamous-cell | | | |
| carcinoma of the head | | | |
| and neck | | | |

Table 3, taken from Turkson J and Jove R 2000, indicates numerous tumours associated directly with the anomalous activation of STAT3.

TABLE 3

| | activated | |
|---|---|---|
| Type of tumour | STATs | References |
| Breast tumours | STAT 1, 3 | (Garcia et al., 2000; Watson and Miller, 1995) |
| Tumours | | (J Bromberg and J E Darnell, unpublished results; |
| | | P Chaturvedi and E P Reddy, unpublished results; |
| | | R Garcia, C Muro-Cacho, S Minton, C Cox, N Ku, R |
| | | Falcone, T Bowman and R Jove, unpublished results) |
| cells | STAT 3 | (Garcia et al., 1997; Sartor et al., 1997) |
| Neck and head tumours | STAT 1, 3 | (Grandis et al., 1998, 2000a) |
| Cell lines and tumours | | |
| Malignant melanomas | STAT 1, 3 | (Florenes et al., 1999; Kirkwood et al., 1999; Pansky |
| Cell lines and tumours | | et al., 2000) |
| Pituitary tumours | STAT 1 | (Ray et al., 1998) |
| Cell lines | | |
| Brain tumours | | |
| (primary tumours | | |
| Gliomas | STAT 1, 3 | (Cattaneo et al., 1998) |
| Medulloblastomas | STAT 3 | (Cattaneo et al., 1998) |
| Brain meningiomas | STAT 1, 3, 5 | (Magrassi et al., 1999; Schrell et al., 1998) |
| Multiple myelomas | STAT 1, | (Catlett-Falcone et al., 1999b) |
| Cell lines and tumours | | |
| Lymphomas | | |
| (cell lines and tumours) | | |
| Large T-cell anaplastic | STAT 3, 5 | (Zhang et al., 1996c) |
| lymphoma | | |
| Sezary syndrome | STAT 3, 5 | (Zhang et al., 1996c) |
| EBV-related/Burkitt's HSV | STAT 3 | (Weber-Nordt et al., 1996) |
| lymphoma | | |
| Saimiri-dependent HSV (T- | STAT 3 | (Lund et al., 1997b, 1999) |
| cell) | | |
| T-cell cutaneous lymphoma | STAT 3 | (Sun et al., 1998) |
| LSTRA T-cell lymphoma | STAT 3 | (Yu et al., 1997) |
| (mouse) | | |
| Mycosis fungoides | STAT 3 | (Nielsen et al., 1997) |
| Leukaemias (tumours and | | |
| cell lines) | | |
| HTLV-I dependents | STAT 3, 5 | (Migone et al., 1995; Takemoto et al., 1997) |
| Chromic lymphocytic | STAT 1, 3 | (Frank et al., 1997) |
| leukaemia (CLL) | | |
| Acute myeloid leukaemia | STAT 1, 3, 5 | (Chai et al., 1997; Gouilleux-Gruart et al., 1996; |
| (AML) | | Weber-Nordt et al., 1996) |
| Megakaryocytic leukaemia | STAT 1, 3, 5 | (Liu et al., 1999) |
| Large granular lymphocytic | STAT 3 | (Epling-Burnette et al., 2000) |
| leukaemia (LGL) | | |
| OTHER TUMOURS | | |
| (tumours and cell lines) | | |
| Prostate | STAT 3 | L Mora, R Garcia, J Seigne, T Bowman, M Huang, G |
| Renal cell carcinoma | STAT 3 | Niu, J Pow-Sang, J Diaz, C Muro-Cacho, D Coppola, |
| Ovarian carcinoma | STAT 3 | T Yeatman, J Cheng, S Nicosia, S Shivers, T |
| Melanoma | STAT 3 | Landowski, D Reintgen, W Dalton, H Yu and R Jove, |
| | | unpublished results |

In particular, in relation to STAT3, the following has been demonstrated in numerous publications:

1) STAT3 is often constitutively active (phosphorylated) in many human cancer cells, such as multiple myeloma, lymphoma, leukaemia, lung cancer, prostate cancer, squamous-cell carcinoma cells of the head and neck, and other tumour types.

2) STAT3 is activated by growth factors (for example EGF, TGF-α, IL-6, IL-10, IL-23, IL-21, IL-11, HGF), kinase oncogenics (for example Src).

3) STAT3 mediates the expression of proliferation genes (for example c-myc, cyclin D1), of apoptosis suppressor genes (for example Bcl-XL and survivin), of cytokine coding genes, and of genes that promote angiogenesis (for example VEGF), increasing, when activated, cell proliferation and angiogenesis and inhibiting apoptosis.

4) The activation of STAT3 also correlates with phenomena of chemoresistance and radioresistance.

5) The persistent activation of STAT3 increases, in various human cancers, proliferation, survival, angiogenesis and metastasis and inhibits anti-tumour immunity.

It is also known that chronic inflammation in certain organs or at certain sites promotes malignant transformation, and that STAT3 is crucial for the extrinsic and intrinsic pathways of inflammations that lead to cancer, STAT3 being known in fact to guide the malignant characteristics associated with chronic inflammation.

Due to the crucial role of STAT3 in tumourigenesis, the inhibitors of STAT3 have enormous potential in the prevention and in the treatment of cancer. Perhaps one of the best-known inhibitors of the activation of STAT3 is AG490, which inhibits the activation of JAK2. Other inhibitors of STAT3 include small peptides, oligonucleotides, and small molecules. Some authors have identified peptides that block the phosphorylation/activation of STAT3, this being a mechanism that mediates the binding to the DNA and the activity of gene regulation, and cell transformation. Various small molecules that block STAT3 include PGJ2, complexes of platinum, ethanol, sodium salicylate, retinoic acid, atiprimod, PS-341 and statins. Many plant polyphenols have been identified for their ability to suppress the activation of STAT3. These include curcumin, resveratrol, cucurbitacin, indirubin, piceatannol, parthenolide, flavopiridol, magnolol, and epigallocatechin-3-gallate. The way in which these molecules succeed in suppressing the activation of STAT3 is not entirely clear. For example, curcumin has demonstrated the effect of inhibition of JAK2, Src, Erb2 and EGFR, which are all involved in the activation of STAT3, also downregulating the expression of Bcl-xL, cyclin D1, VEGF, and TNF, of which the expression is regulated by STAT3 (Aggarwal B. B. et al. Ann. N.Y. Acad. Sci. 1091; 151-69: 2006).

There are thus various strategies and various mechanisms that make it possible to intervene in the cascade of signalling of STAT3: inhibiting the phosphorylation/activation of STAT3, inhibiting the intermolecular interactions that involve STAT3, inhibiting the nuclear import/export of STAT3, inhibiting the transcription mediated by STAT3. Apart from the chemotherapeutic agents already mentioned that inhibit STAT3, there are also others (cetuximab, gefitinib, erlotinib, etc.), for which different effects have been reported: a modest efficacy, the development of resistances, myelosuppression, toxicity at gastro-intestinal level, and various adverse events including cardiovascular toxicity (see Table 4).

Table 4, below, taken from Johnston P A and Grandis R G 2011, reports strategies and results for the therapeutic intervention of the signal of STAT3.

TABLE 4

| Strategy | Target | Examples | Results |
|---|---|---|---|
| Inhibition of the | EGFR | Cetuximab, panitumumab, | Modest efficacy; |
| phosphorylation/activation | competitiveness | Gefitinib, erlotinib, | development of |
| of STAT3 | Activity TKR | lapatinib, AG490, LS-104, | resistances; |
| | Activity JAK | ICNB1824, CEP-701, | myelosuppression; |
| | Activity SFK | Dasatinib, AZD0530, | gastro-intestinal (GI) |
| | | basutinib | toxicity and adverse |
| | | | effects; kinase selectivity |
| | | | and cardiovascular |
| | | | toxicity |
| Inhibit the intermolecular | SH-2 domains of | Designated oligopeptides | Scarce cell permeability |
| interactions that involve | STAT3 | from EGFR, gp130, and | and efficacy; scarce |
| STAT3 | | other receptors or peptides | metabolic stability; |
| | | containing pY; octamer | scarce selectivity for |
| | | peptides quartet of G | specific SH2 domains; |
| | | oligonucleotides; small | potential adverse events |
| | | peptidomimetic molecules | |
| Inhibit the nuclear | Imports α3, α5, α7 | Karyostatin 1A (non- | Multi-component nature |
| import/export of STAT3 | Import β | determined effects on | of the nuclear pore and |
| | Export 1 | STAT3), Leptomycin B and | incompletely determined |
| | | Ratjadone A | translocation; |
| | | | problematic specificity for |
| | | | the translocation of the |
| | | | proteins |
| Inhibition of STAT3- | Not specified | dsODNdecoy; octamer | Scarce cell permeability |
| mediated transcription | | peptides | without effective and |
| | | | specific distribution |
| | | | systems; scarce |
| | | | metabolic stability |
| Natural products | Not specified | Guggulsterone, honokiol, | Unknown specificity, |
| | | curcumin, resveratrol, | power, efficacy and |
| | | flavopiridol, cucurbitacin | mechanism of action |

Therapies that are targeted therapies by means of compounds that inhibit a specific target molecule in a more specific manner, in sub-populations of cells directly involved in tumour progression, represent a new perspective in the treatment of cancer. The molecules that control cell proliferation and death, such as receptor tyrosine kinases (RTKs) for growth factor are among the best objectives of this type of therapeutic approach. The era of targeted therapy started with the approval of trastuzumab, a monoclonal antibody against HER2, for the treatment of metastatic mammary carcinoma and imatibin, an inhibitor of BCR-ABL, in chronic myeloid leukaemia. In spite of the initial enthusiasm for the efficacy of these treatments, the doctors had to immediately confront the problem of relapse, since those suffering from cancer almost always developed a resistance to the drugs, often due to the activation of alternative pathways. Since the tumour is characterised by more mechanisms and more gene targets, which are frequently deregulated, it would be advantageous to adopt a combination therapy, as is standard in the treatment of cancer, since this results in a rational strategy for increasing the response and the tolerability and for decreasing resistance. There is currently a rise in interest for the combination of anti-tumour drugs that aim to maximise efficacy, minimising the systemic toxicity by means of the use of lower drug doses.

Thus, pharmacologically safe and effective therapeutic agents, such as molecules of natural origin, which can block constitutive or inducible activation of STAT3, have a potential efficacy in the treatment of cancer, given that more and more tests are concluding that the inhibition of the phosphorylation of STAT3 by means of a pharmacological blocking of the molecules upstream, including Src and JAK, can reduce the formation of tumours, also leading to the possibility of reduction of the necessary dosage of chemotherapeutic drug.

In addition, since the activation of STAT3 also correlates with the resistance to chemotherapy and radiotherapy, inhibitors of such activation are also of great interest for limiting such resistance and optimising the effect of chemotherapy and of radiotherapy.

### SUMMARY OF THE INVENTION

The authors of the present invention have demonstrated that extracts of artichoke (Cynara scolymus) are able to selectively modulate, essentially inhibit, the phosphorylation of the protein STAT3, consequently preventing the subsequent action within the cell as transcription factor. As will be seen in the experimental part of the application, the authors of the invention have demonstrated, in numerous experiments and on various cell lines, that the extracts described here are effective inhibitors of the activation (phosphorylation) of STAT3 and consequently
demonstrate effective cytotoxic action on tumour cell lines,
are able to inhibit the regeneration of tumour cells, thus acting as cytostatics,
induce apoptosis in tumour cells
have additive and also synergistic effects with numerous chemotherapeutic agents, thus resulting in a reduction of the vitality (viability) of the tumour cells compared with those treated with the chemotherapeutic agent alone or with the extract alone
act in a differential manner on malignant pleural mesothelioma cells and on untransformed mesothelial cells.

The authors of the present invention have also characterized the extract, titrating it for some components, and have then isolated different fractions of extract of Cynara scolymus and titrated them for the same components in order to be able to identify, on the one hand, individual fractions with titrations similar to those of the extract of Cynara scolymus used in the reported experiments, and also so as to be able to mix different fractions among them or with said extract so as to obtain an end compound with titrations similar to those of the extract reported in the examples and in the figures, in order to be able to provide standardized preparations suitable for a clinical use.

From the viewpoint of the effect of such extracts on the STAT3 factor, the authors of the present invention have also demonstrated by way of experiment that the extracts of Cynara scolymus are able to prevent the binding of STAT3 to the DNA and thus to prevent the alteration of the expression of the genes normally activated by phosphorylated STAT3.

In other words, said extract has proven to be capable of modulating, essentially inhibiting, the protein STAT3 in its phosphorylated form, preventing the successive action of said protein within the cell as transcription factor. In particular, the inventors of the present disclosure have demonstrated that an extract of Cynara scolymus is able to inhibit the constitutive or anomalous activation of STAT3 and to induce the reactivation of apoptosis in cultures of MPM tumour cells. In addition, the authors of the present invention have also demonstrated that, in experiments on cultures of MPM tumour cells, the extract of Cynara scolymus inhibits wound healing, in fact preventing the invasivity of the tumour cells. In addition, the authors of the present invention have also demonstrated with experiments of engraftment of tumour cells in mice that the extract of the present invention exerts in vivo an anti-tumour effect with respect to MPM cells.

A first subject of the present invention is therefore an extract of Cynara scolymus or a fraction of extract of Cynara scolymus or a mixture of said extract with one or more of said fractions or a mixture of said fractions, wherein total caffeoylquinic acids represent from 8% to 16% by weight of said extract or of said fraction or of said mixture in dry form, chlorogenic acid represents from 3.5% to 7% by weight of said extract or of said fraction or of said mixture in dry form, and said cynaropicrin represents from 0.2% to 4% by weight of said extract or of said fraction or of said mixture in dry form, for use in the prevention and/or in the treatment of an inflammatory and/or pre-tumour and/or tumour pathological condition characterised by a constitutive or anomalous activation of the STAT3 transcription factor, wherein the extract, the fraction or the mixtures are used in association with one or more active compounds with anti-tumour and/or anti-inflammatory activity.

A second subject of the present invention is a composition comprising or consisting in an extract of Cynara scolymus or a fraction of extract of Cynara scolymus or a mixture of said extract with one or more of said fractions or a mixture of said fractions, wherein total caffeoylquinic acids represent from 8% to 16% by weight of said extract or of said fraction or of said mixture in dry form, chlorogenic acid represents from 3.5% to 7% by weight of said extract or of said fraction or of said mixture in dry form, and said cynaropicrin represents from 0.2% to 4% by weight of said extract or of said fraction or of said mixture in dry form, in association with one or more agents with anti-tumour activity and a carrier and/or diluent and/or excipient for use in the prevention and/or in the treatment of an inflammatory and/or pre-tumour and/or tumour pathological condition characterised by a constitutive or anomalous activation of the STAT3 transcription factor.

A third object of the present invention is a kit for concomitant or sequential administration of an extract of Cynara scolymus or a fraction of extract of Cynara scolymus or a mixture of said extract with one or more of said fractions or a mixture of said fractions, wherein total caffeoylquinic acids represent from 8% to 16% by weight of said extract or of said fraction or of said mixture in dry form, chlorogenic acid represents from 3.5% to 7% by weight of said extract or of said fraction or of said mixture in dry form, and said cynaropicrin represents from 0.2% to 4% by weight of said extract or of said fraction or of said mixture in dry form, and of one ore more compounds with anti-inflammatory and/or anti-tumour activity (anti-inflammatory and/or anti-tumour compounds), comprising one or more aliquots of an extract of Cynara scolymus or a fraction of extract of Cynara scolymus or a mixture of said extract with one or more of said fractions or a mixture of said fractions as above defined or one or more aliquots of a composition comprising, as active pharmaceutical ingredient, an extract of Cynara scolymus or a fraction of extract of Cynara scolymus or a mixture of said extract with one or more of said fractions or a mixture of said fractions as defined above and one or more separate aliquots of a chemotherapeutic agent or a mixture of chemotherapeutic agents with suitable pharmaceutically acceptable carriers for use in the prevention and/or in the treatment of an inflammatory and/or pre-tumour and/or tumour pathological condition characterised by a constitutive or anomalous activation of the STAT3 transcription factor in association with a chemotherapeutic agent.

A fourth subject of the invention is a therapeutic method for the prevention and/or in the treatment of an inflammatory and/or pre-tumour and/or tumour pathological condition characterised by a constitutive or anomalous activation of the STAT3 transcription factor comprising the step of administering to an individual who needs it a therapeutically active quantity of an extract of Cynara scolymus or a fraction of extract of Cynara scolymus or a mixture of said extract with one or more of said fractions or a mixture of said fractions, wherein total caffeoylquinic acids represent from 8% to 16% by weight of said extract or of said fraction or of said mixture in dry form, chlorogenic acid represents from 3.5% to 7% by weight of said extract or of said fraction or of said mixture in dry form, and said cynaropicrin represents from 0.2% to 4% by weight of said extract or of said fraction or of said mixture in dry form, or of a pharmaceutical composition as above defined.

For the purposes of the present description, the term Cynara scolymus corresponds to the term Cynara cardunculus subsp. scolymus and can be substituted therewith in any point of the description and of the claims.

For the purposes of the present description, the term “comprising” can be substituted in any point of the description and of the claims with the term “consisting of”.

### DETAILED DESCRIPTION OF THE FIGURES

Note: In the present figures, the extract of Cynara spp. used is often indicated by the abbreviation ABO-1.

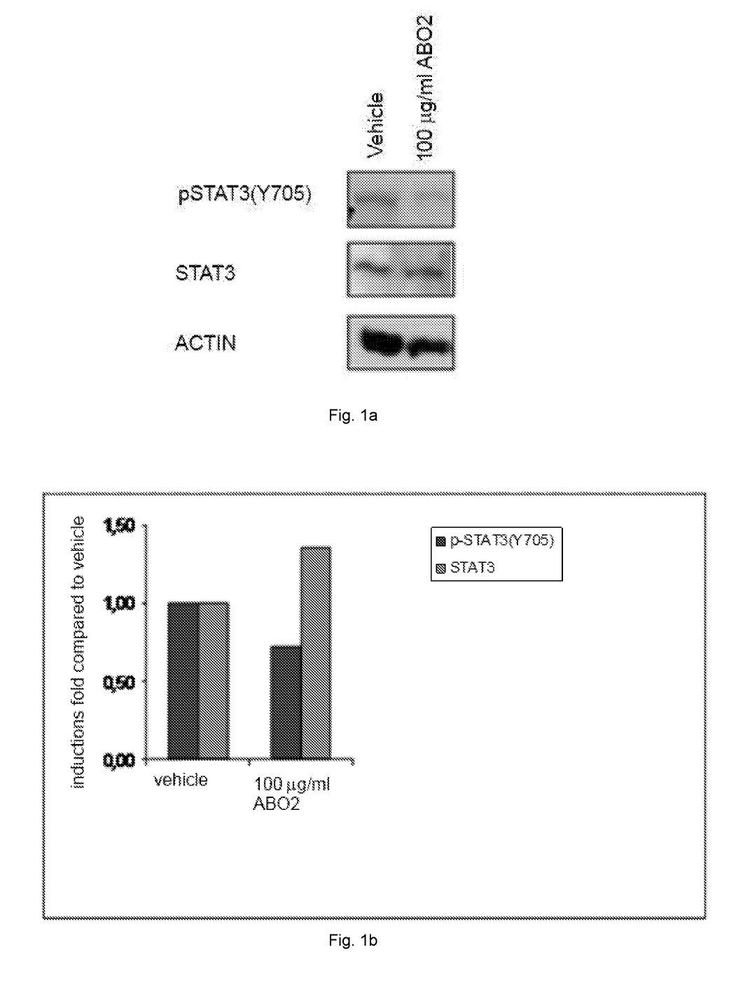

FIG. 1: Inhibition of the phosphorylation of STAT3, p-STAT3 (Y705)

FIG. 1A Western Blot analyses of cell lysates obtained from MSTO211H treated with 100 μg/ml of Cynara scolymus extract for 24 hours. Quantification was performed compared with a control of Actin.

FIG. 1B Bar chart of the data obtained with Western Blot on MSTO211H cells. p-STAT3 (phosphorylated STAT3) is shown in black, STAT3 is shown in grey.

The figure shows that the extract inhibits the formation of p-STAT3 compared with the control.

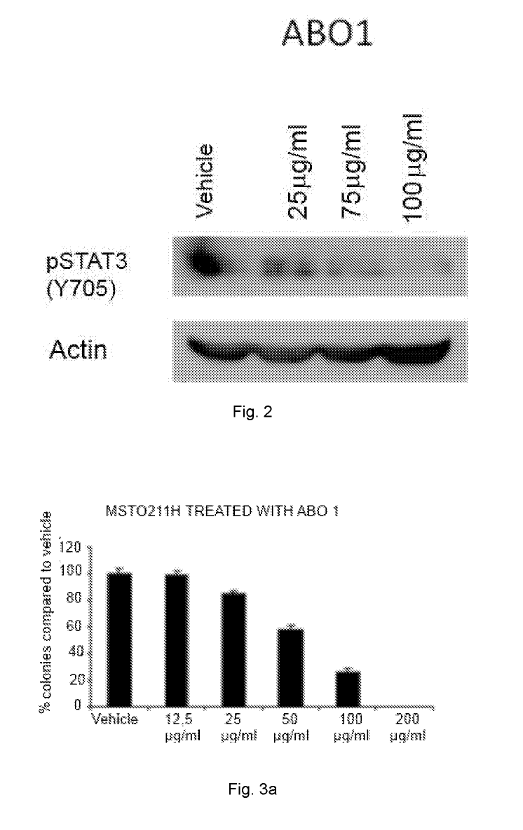

FIG. 2: Western Blot analyses of cell lysates of MSTO211H cells treated with 25-50-75 μg/ml of Cynara scolymus extract in the p-STAT3 row, with the Actin control below. The figure shows that the extract inhibits STAT3 phosphorylation and that this inhibition is dose-dependent.

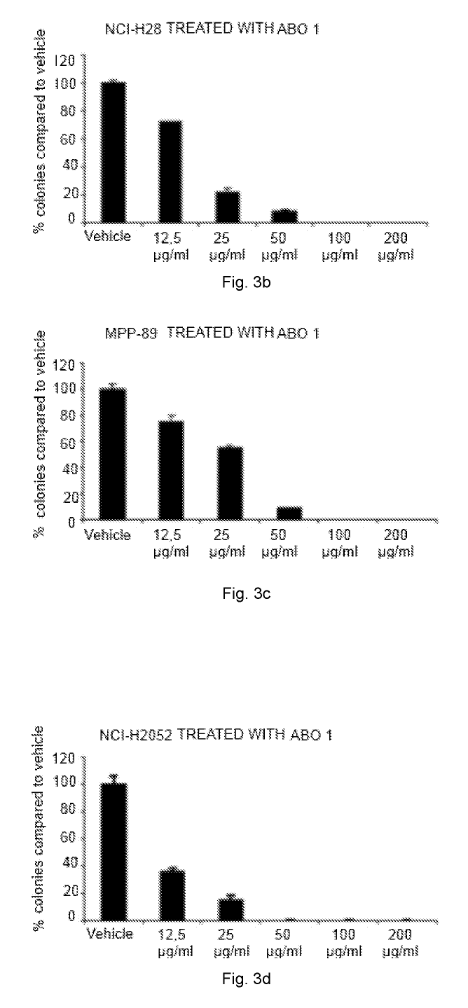

FIG. 3: Clonogenic assay (see the experimental section for the conditions) on cell lines of human malignant pleural mesothelioma with various doses of extract of Cynara scolymus

graph 3a. assay performed on human mesothelioma cell line MSTO211H
graph 3b. assay performed on human mesothelioma cell line NCI-H28
graph 3 c. assay performed on human mesothelioma cell line MPP-89
graph 3d. assay performed on human mesothelioma cell line NCI-H2052

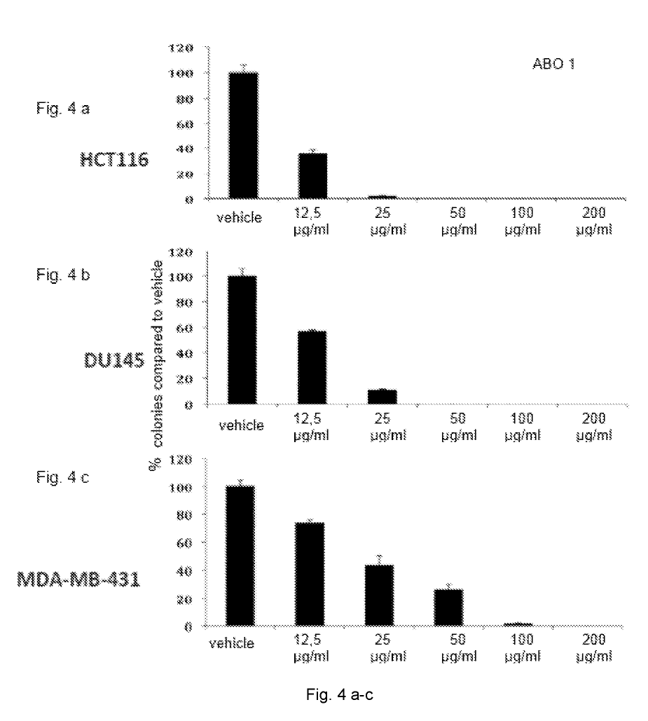

FIG. 4: The extract of the invention influences the ability of 3 different inflammatory tumour lines (HCT116, MDA-MB-231 E DU145) to form colonies, in a dose-dependent manner, independently of the isotypes thereof.

graph 4a. assay performed on colon tumour cell line HCT116
graph 4b. assay performed on prostate tumour cell line DU145
graph 4c. assay performed on breast tumour cell line MDA-MB-231

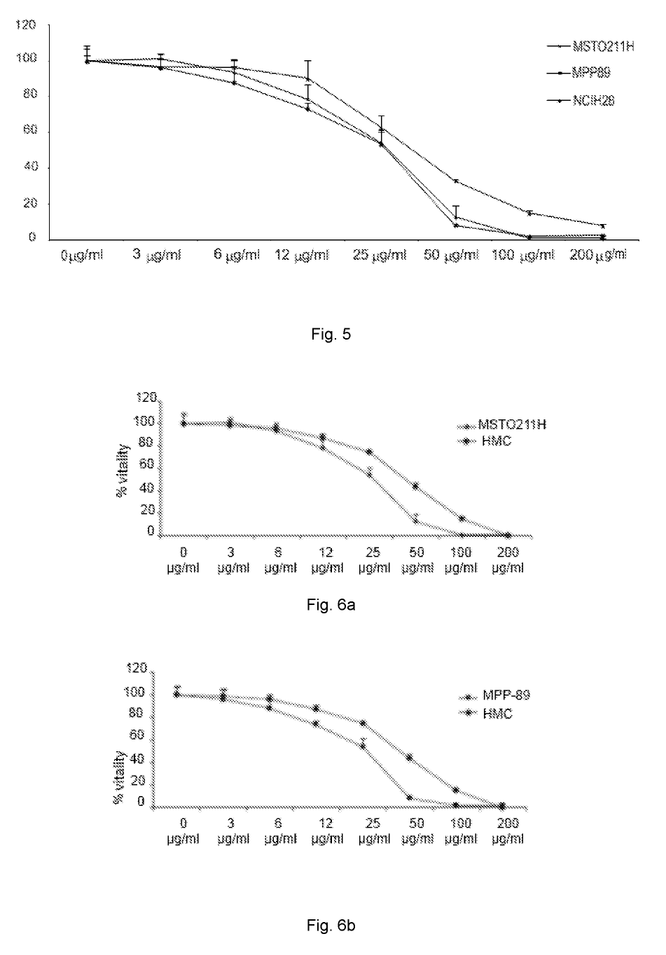

FIG. 5: Assay of cell vitality using ATPlite assay (see the experimental section for the conditions) on malignant pleural mesothelioma cell lines (MSTO211H, MPP-89, NCI-H28). The assay shows that cell vitality is inhibited by the extract of Cynara scolymus of the invention in a dose-dependent manner in various mesothelioma cell lines.

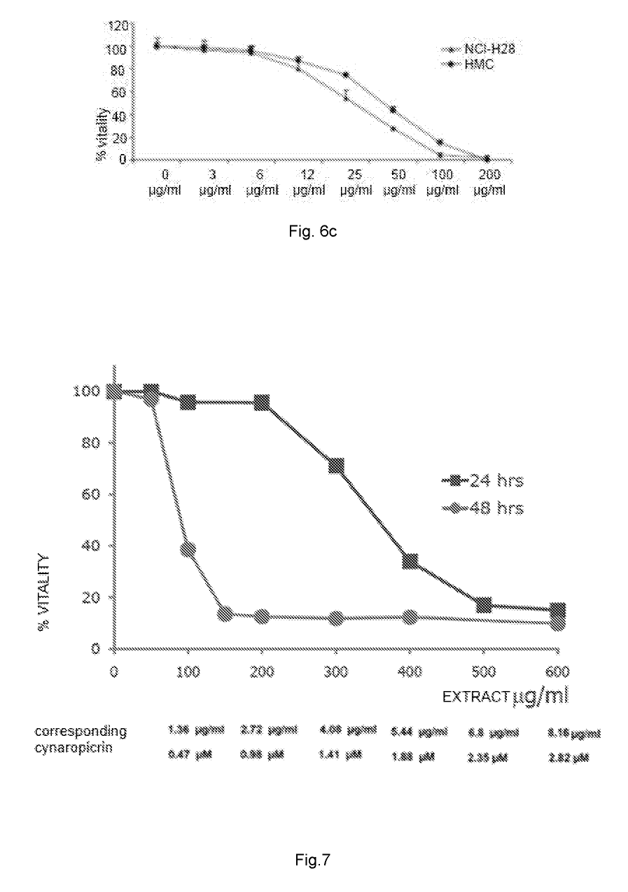

FIG. 6: comparison of the three vitality curves of FIG. 5 compared with (FIG. 6a MSTO211H, FIG. 6b MMP-89, FIG. 6c NCI-H28) the proliferation curve obtained treating normal mesothelioma cells (HMC) with extract of Cynara scolymus. The malignant mesothelioma cell lines (MPMs) clearly show the anti-proliferative effect of the extract of Cynara scolymus compared with the HMCs.

FIG. 7 Assay of cell vitality in the confluent prostatic adenocarcinoma cell line DU145, treated with various concentrations of artichoke extract (50-600 μg/ml) for various treatment times (24 and 48 hours) with indications of the content in cynaropicrin of the extract. The confluency of the cells increases the levels of constitutively activated STAT3, making the cells themselves largely resistant to death. The vitality was analysed using the WST-1 assay (WST-1 test, see the experimental section for the conditions). The figure shows that the extract inhibits vitality in a time-dependent and dose-dependent manner. The squares show the trend over 24 hours and the circles show the trend at 48 hours with extract doses from 0 to 600 μg/ml and the respective content in cynaropicrin, expressed both in μg/ml and in μM, of the extract at the various concentrations (100, 200, 300, 400, 500, 600 μg/ml).

Cells with high levels of activation of STAT3: the results obtained show that the extract inhibits cell vitality with EC₅₀=380 microg/ml at 24 hours and EC₅₀=100 μg/ml at 48 hours.

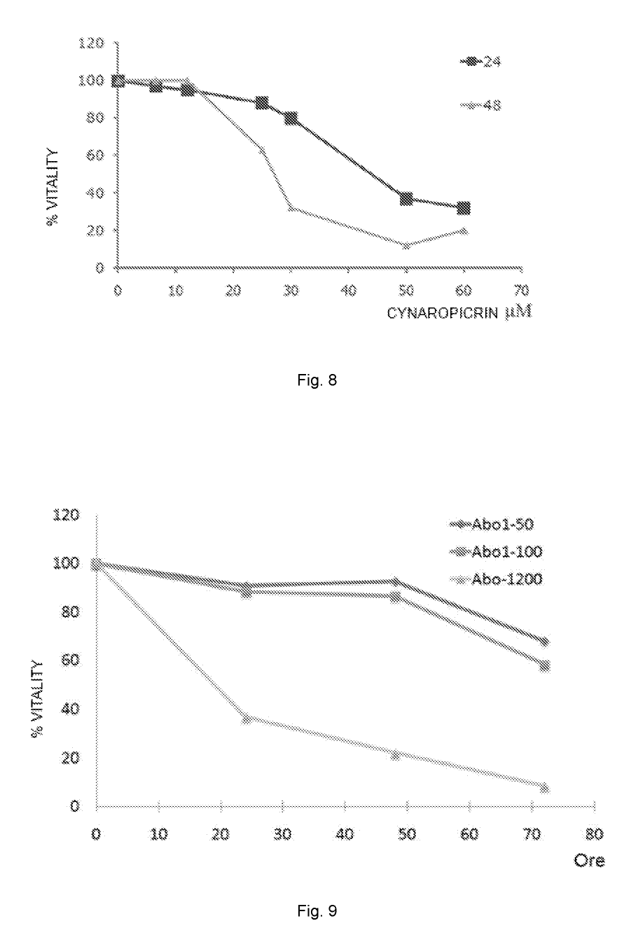

FIG. 8. Assay of cell vitality in the confluent prostatic adenocarcinoma cell line DU145, treated with various concentrations of cynaropicrin (0-70 μm) for various treatment times (24 or 48 hours). The confluency increases the levels of constitutively activated STAT3, making the cells largely resistant to death. The vitality was analysed using the WST-1 assay (WST-1 test, see the experimental section for the conditions). The figure shows that cynaropicrin inhibits cell vitality in a time-dependent and dose-dependent manner. The squares show the trend at 24 hours and the triangles show the trend at 48 hours with different concentrations: 10, 20, 30, 40, 50, 60 μM of cynaropicrin.

The data presented show that cynaropicrin is less effective than the artichoke extract. The figure shows that cynaropicrin inhibits cell vitality and proliferation in a time-dependent and dose-dependent manner much less effectively compared with the artichoke extract (see FIG. 8 as a comparison). For example: to have an effect of reduction of vitality equal to approximately 90%, treatments with 50 μM for 48 hours, compared with 0.94-2.82 μM, are necessary when cynaropicrin is contained within the lyophilised extract.

FIG. 9. Assay of cell vitality in the non-confluent cell line DU145, thus with low levels of constitutively activated STAT3, treated with various concentrations of artichoke extract and for various treatment times (24-48-72 hours). The vitality was analysed using the WST-1 assay (test WST-1, see the experimental section for the conditions). The circles denote a treatment with 50 μg/ml of Cynara scolymus, the squares a treatment with 100 μg/ml, and the triangles a treatment with 200 μg/ml. The figure shows how the cell vitality of the cell line DU145 is highly compromised by Cynara scolymus 200 μg/ml. The results obtained show that 200 p/ml of extract inhibit cell vitality by 60% at 24 hours. As can be seen, compared with FIG. 8, with respect to experiments on cells with high levels of activation of STAT3, the EC₅₀ of this experiment are considerably lower (approximately 200 vs 380 μg/ml at 24 hours), thus demonstrating a greater power of the extract of the invention in cells with low level of activation of STAT3 (non-confluent). Such experiments thus confirm that the cells in which STAT3 is active have a greater degree of malignancy. The inhibition of the phosphorylation of STAT3 is the primary mechanism of reduction of cell vitality.

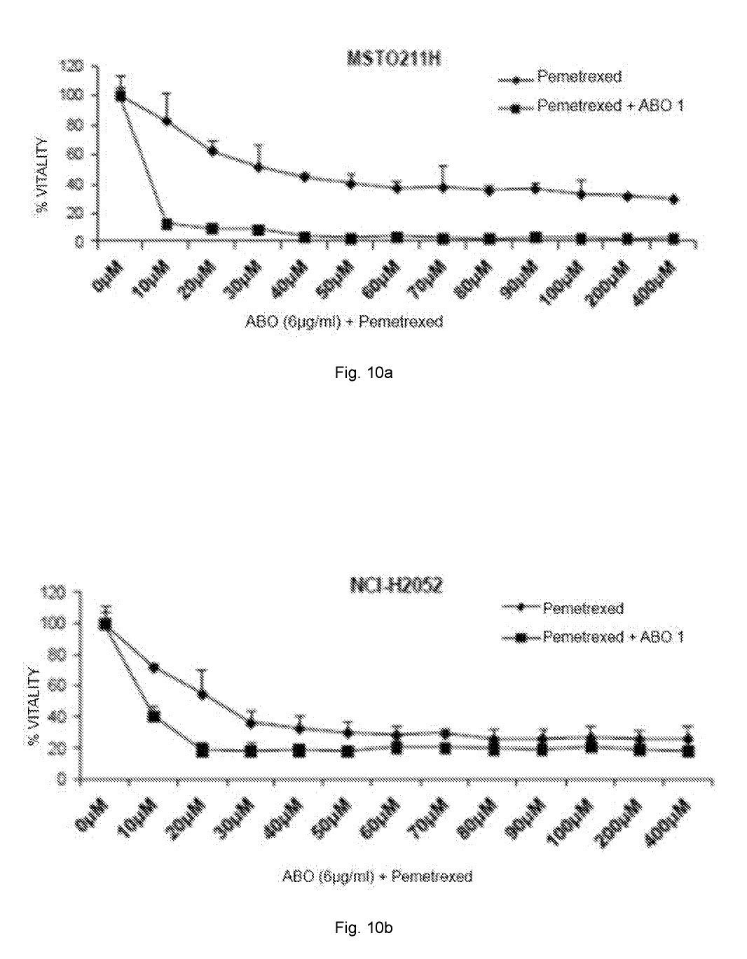

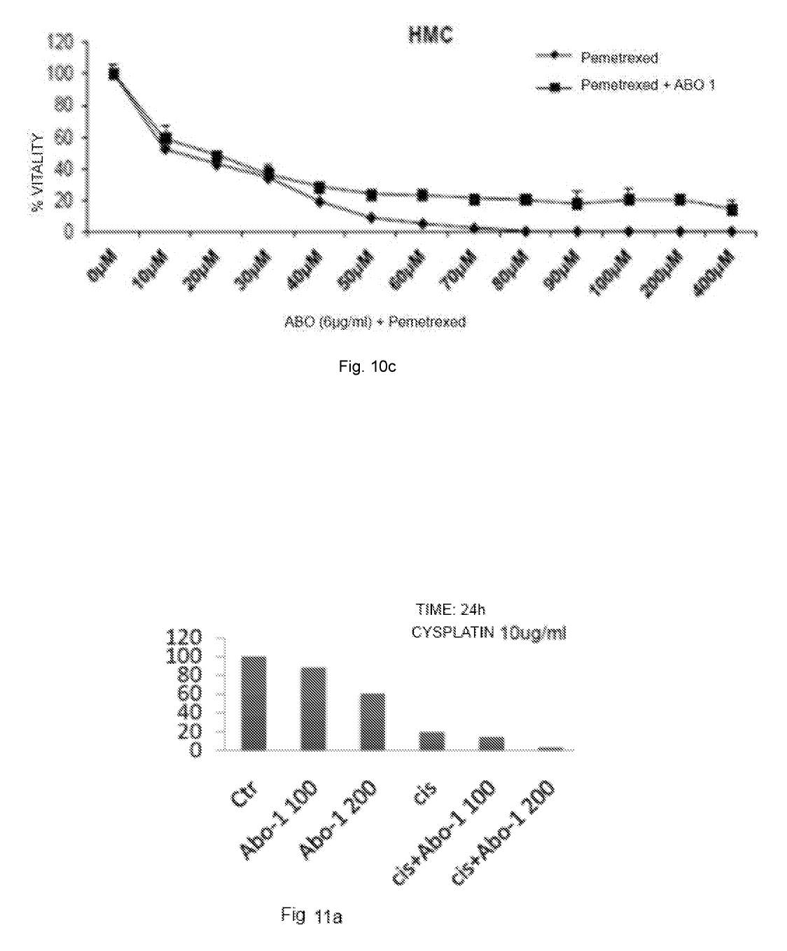

FIG. 10: Assay of cell vitality (ATPlite assay) following treatment with artichoke extract in association with pemetrexed (PMTX) on mesothelioma cell lines MPM (FIG. 10a MSTO211H and FIG. 10b NCI-H2052) and transformed on mesothelioma cells (FIG. 10c HMC). The treatment with PMTX is cytotoxic for the MPM cells and highly toxic for the non-tumour cells. The co-treatment of the cells with the extract of the invention+PMTX had a significant effect on cell vitality in MPM cell lines, whilst reducing the mortality caused by pemetrexed in the untransformed cells (HCM). Consequently, it is clear that the extract of artichoke of the invention makes only the tumour cells sensitive to pemetrexed.

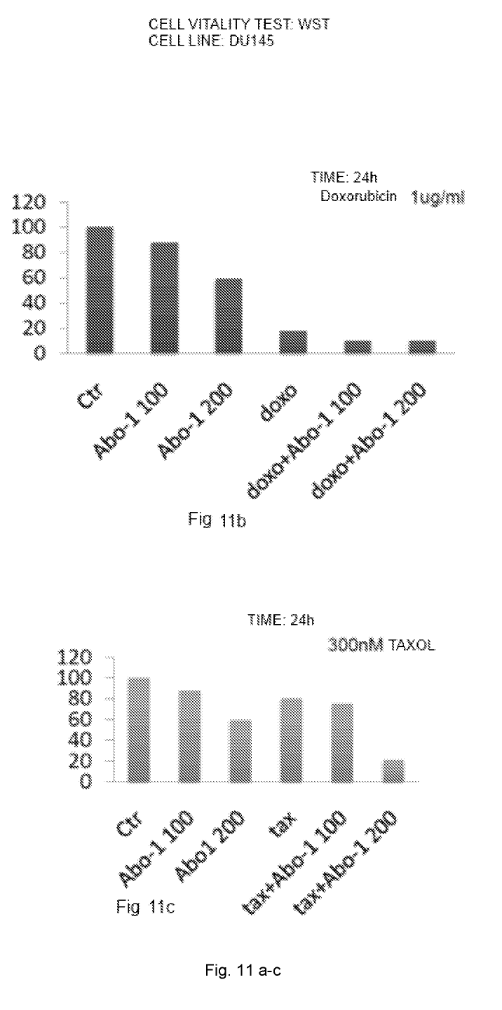

FIG. 11 Cell vitality assay WST-1 following treatment with extract of artichoke in association with various chemotherapeutic agents: doxorubicin, taxol, cisplatinum (see experimental section for the conditions) on a human prostate tumour cell line DU145. The vitality was analysed using the WST-1 assay (test WST-1, see the experimental section for the conditions). FIG. 11 shows the cell vitality following treatment for 24 hours, with two different doses of artichoke extract (100 and 200 μg/ml), with just cisplatinum at 10 μg/ml and with artichoke extract (100 and 200 μg/ml) in association with cisplatinum at 10 μg/ml.

FIG. 11b shows cell vitality following treatment for 24 hours, with two different doses of artichoke extract (100 and 200 μg/ml), with doxorubicin at 1 μg/ml and of artichoke extract (100 and 200 μg/ml) in association with doxorubicin at 1 μg/ml on human carcinoma cells DU145.

FIG. 11c shows the cell vitality following treatment for 24 hours, with two different artichoke extracts (100 and 200 μg/ml), with taxol 300 nM, and artichoke extract (100 and 200 μg/ml) in association with taxol 300 nM on human carcinoma cells DU145.

In all the experiments the extract forming the basis of the invention enhances the cytotoxicity of the three chemotherapeutic agents with a greater efficacy in the case of cisplatinum.

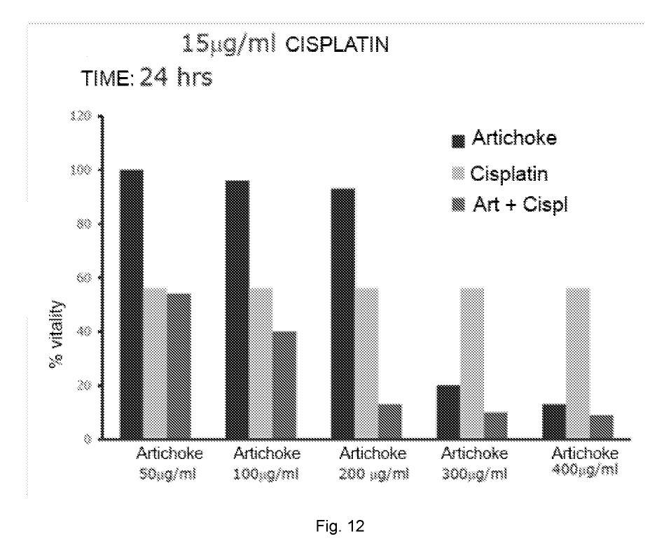

FIG. 12. Assay of cell vitality after treatment with associations of artichoke extract and cisplatinum on human carcinoma cells DU145 (see experimental section for the conditions). The figure shows the comparison between treatments with artichoke extract (black), cisplatinum (light grey) and artichoke+cisplatinum (white) at various concentrations of artichoke extract and at fixed concentration of 15 μg/ml of cisplatinum.

The relative concentrations of cynaropicrin are shown in the figure.

The extract forming the basis of the invention enhances the cytotoxicity of cisplatinum with a greater effect at the dose of 200 μg/ml.

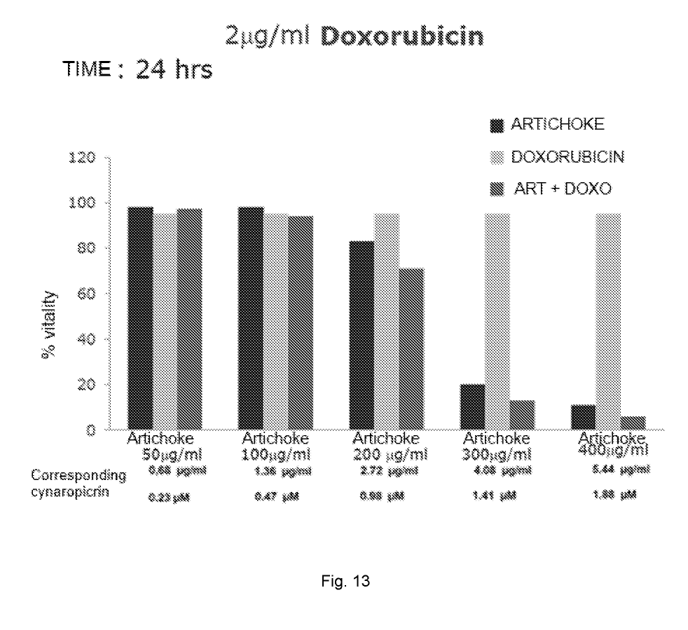

FIG. 13. Vitality assay after treatment with association of artichoke extract and doxorubicin on human carcinoma cells DU145 (see experimental section for the conditions). The figure shows the comparison between treatments with artichoke extract (black), doxorubicin (light grey), and artichoke+doxorubicin (white) at various concentrations of artichoke extract and at fixed concentration of 2 μg/ml of doxorubicin.

The relative concentrations of cynaropicrin are the same as reported in FIG. 13.

The extract forming the basis of the invention enhances the cytotoxicity of doxorubicin with a greater effect at the dose of 200 μg/ml.

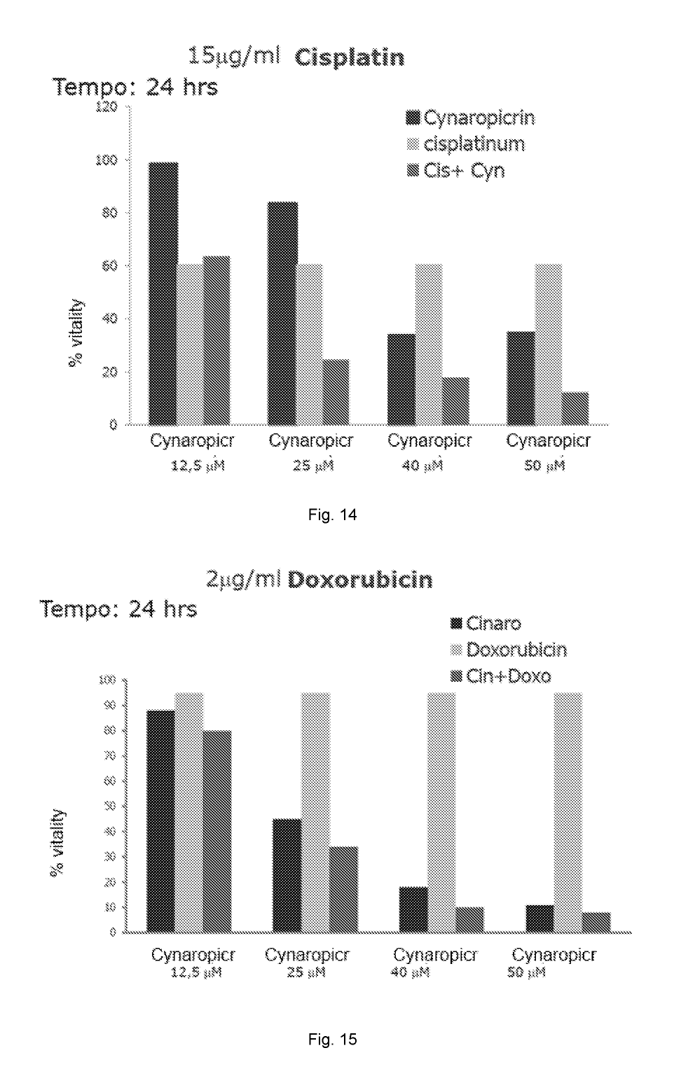

FIG. 14. Vitality assay after treatment with association of cynaropicrin and cisplatinum on human carcinoma cells DU145 (see experimental section for the conditions). The figure shows the comparison between treatments with cynaropicrin (black), cisplatinum (light grey), and cynaropicrin+cisplatinum (white) at various concentrations of cynaropicrin and at fixed concentration of 15 μg/ml of cisplatinum.

It would appear that, to obtain an effect that reduces cell vitality below 20%, a molarity of cynaropicrin forty times greater than that present in the artichoke extract is necessary (see FIG. 12).

FIG. 15 Vitality assay after treatment with association of cynaropicrin and doxorubicin on human carcinoma cells DU145 (see experimental section for the conditions). The figure shows the comparison between treatments with cynaropicrin (black), doxorubicin (light grey), and cynaropicrin+doxorubicin (white) at various concentrations of cynaropicrin and at fixed concentration of 2 μg/ml of doxorubicin.

It would appear that, to obtain an effect that reduces the cell vitality below 20%, a molarity of cynaropicrin approximately twenty-five times greater than that present in the artichoke extract is necessary (see FIG. 13).

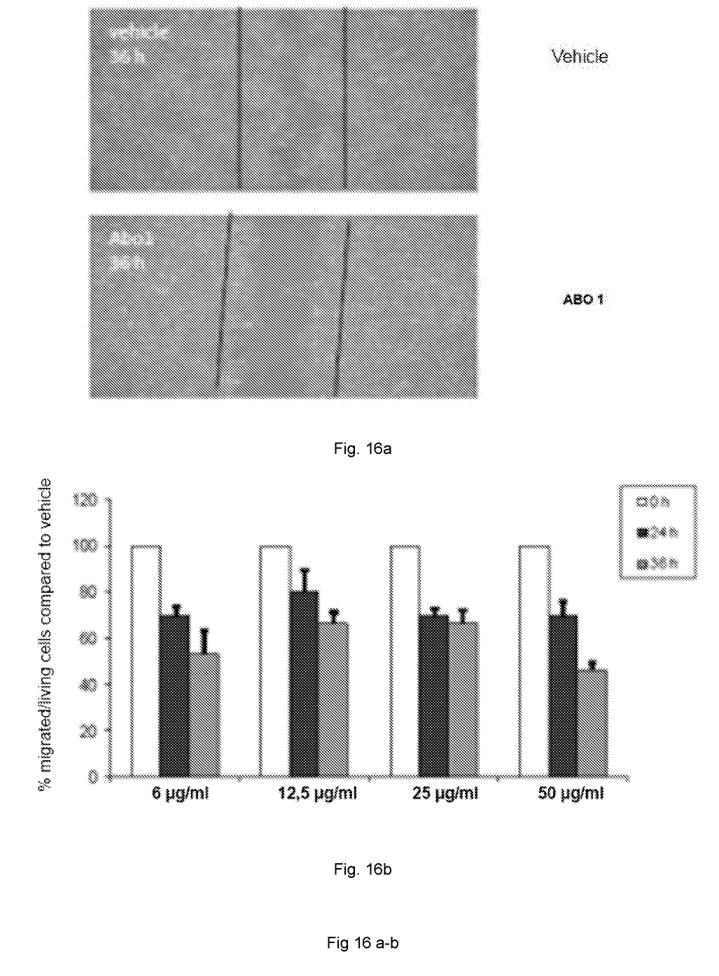

FIG. 16: Assays of wound healing on human mesothelioma cell line MSTO221H (see experimental section for the conditions).

Graph 16a shows the wound healing at 36 h in control plates with just the carrier (vehicle) and with product at a concentration of 6 μg/ml, whereas image 16b shows bar charts concerning the efficacy in closing the wound (quantification of the number of cells in %) treated with the extract of the invention and with carrier at the times indicated.

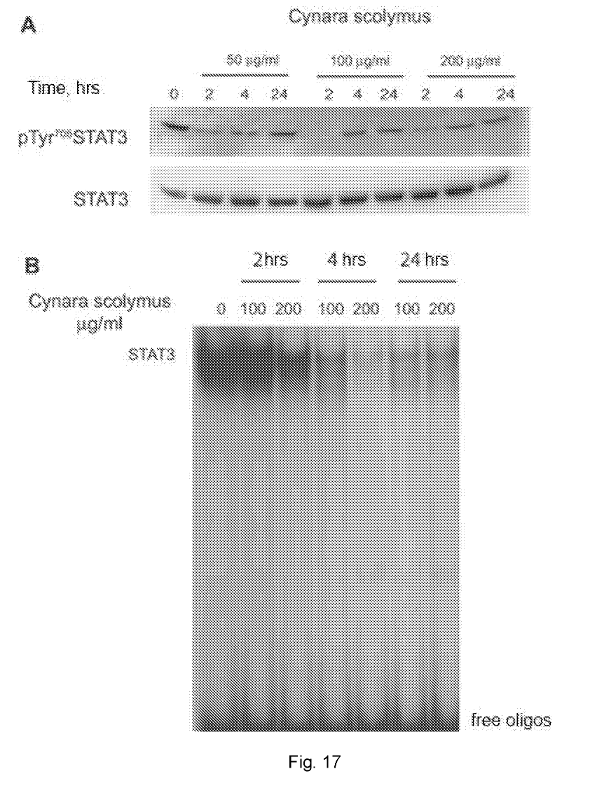

FIG. 17: The extract of Cynara Scolymus modulates the pathway of STAT3 in DU145 cells: in particular, the figure shows that the extract inhibits the constitutive activation of STAT3 in DU145 cells and also inhibits the binding of STAT3 to DNA.

17a) Western Blot: the extract of Cynara scolymus (200 μg/ml) inhibits the phosphorylation of STAT3 after 2-4 hours of treatment without modifying the expression of the protein.
17b) EMSA: the extract of Cynara scolymus (200 μg/ml) inhibits the binding of STAT3 to DNA after 2-4 hours of treatment in the DU145 cell line (FIG. 17b).

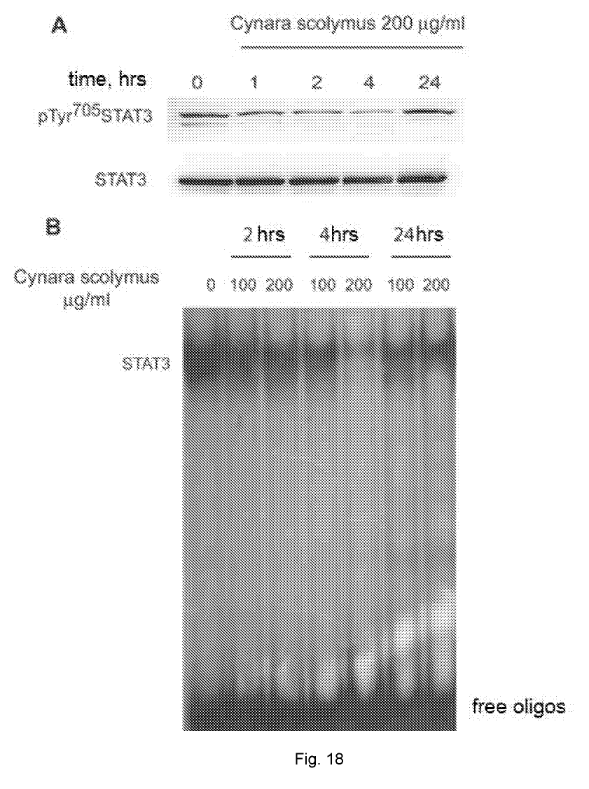

FIG. 18: The extract of Cynara Scolymus modulates the pathway of STAT3 in KARPAS cells: in particular, the figure shows that the extract inhibits the constitutive activation of STAT3 in KARPAS cells and also inhibits the binding of STAT3 to DNA.

18a) Western Blot: the extract of Cynara scolymus (200 μg/ml) inhibits the phosphorylation of STAT3 after 2-4 hours of treatment without modifying the expression of the protein in the KARPAS cell line.
18b) EMSA: the extract of Cynara scolymus (200 μg/ml) inhibits the binding of STAT3 to DNA after 2-4 hours of treatment in the cell line KARPAS.

The extract of Cynara scolymus used contains 0.181% of cynaropicrin, thus 200 μg/ml of extract contain 1.2 μM of cynaropicrin.

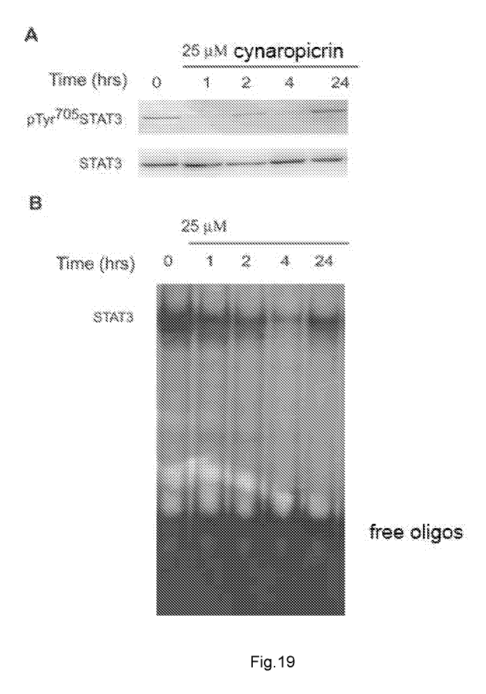

FIG. 19: cynaropicrin modulates the pathway of STAT3 in DU145 cells.

Cynaropicrin inhibits in DU145 cells both the phosphorylation of STAT3 and the ability thereof to bind to DNA with EC₅₀=25 μM (25 μM cynaropicrin=approximately 0.74 μg/ml)

Western Blot: 25 μM of cynaropicrin inhibit the phosphorylation of STAT3 in DU145 cells (FIG. 19a)

EMSA: 25 μM of cynaropicrin inhibit the binding of STAT3 to DNA in DU145 cells (FIG. 19b)

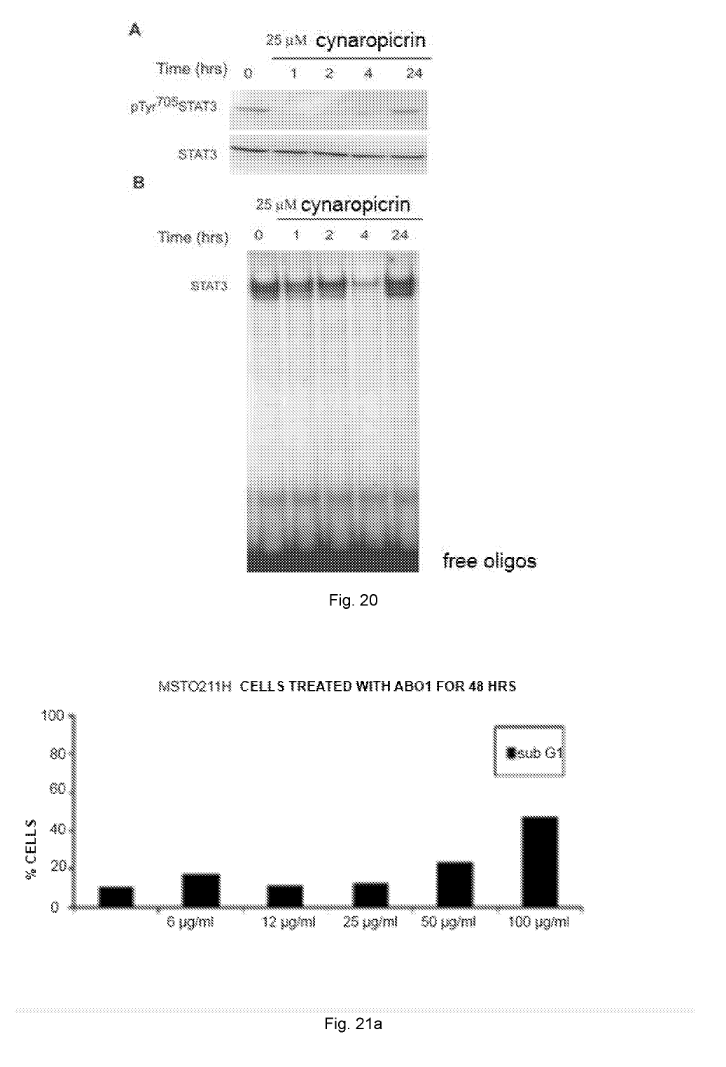

FIG. 20: cynaropicrin modulates the pathway of STAT3 in KARPAS cells.

Cynaropicrin inhibits in KARPAS cells both the phosphorylation of STAT3 and the ability thereof to bind to DNA with EC₅₀=25 μM (25 μM cynaropicrin=approximately 0.74 μg/ml)

Western Blot: cynaropicrin (25 μM) inhibits the phosphorylation of STAT3 in DU145 cells (FIG. 19a)

EMSA: cynaropicrin (25 μM) inhibits the binding of STAT3 to DNA in DU145 cells (FIG. 19b).

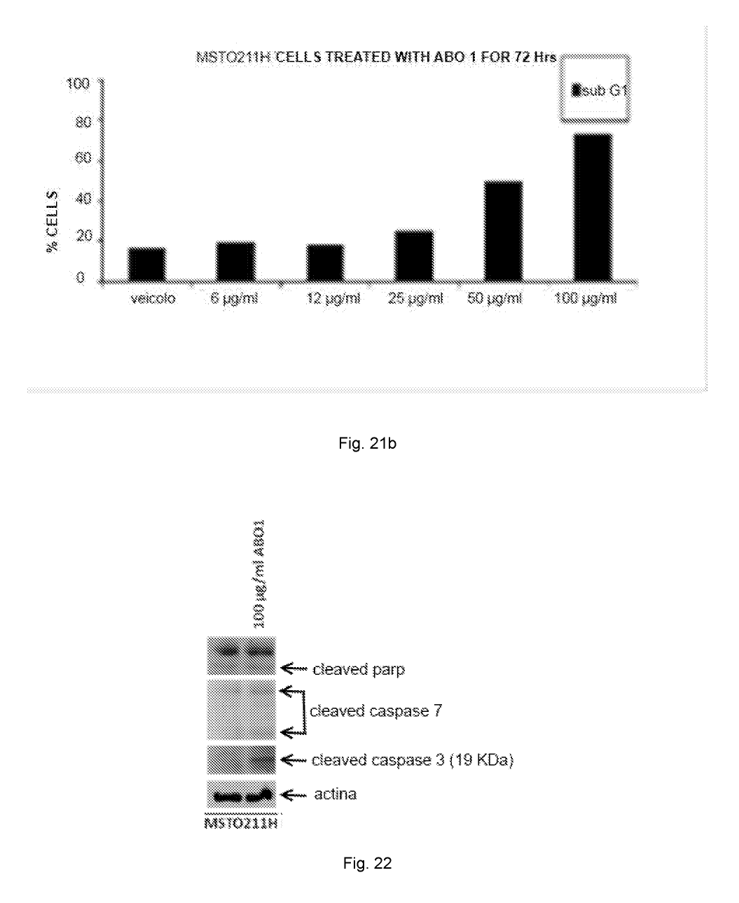

FIG. 21: assessment of the impact of the extract of Cynara scolymus on the cell cycle (FACS method). The extract induces the death of the MPM cells (MSTO211H) by means of an increase in the % of cells in sub G1 phase, both after treatment for 48 hours (FIG. 21a) and after treatment for 72 hours (FIG. 21b)

FIG. 22 Assay to assess the induction of apoptosis (Western method). The extract of the invention at the dose of 100 μg/ml induces apoptosis as demonstrated by the rise in the levels of some apoptotic markers as the cleaved form of PARP and of caspases 3 and 7 in the cell line MSTO211H.

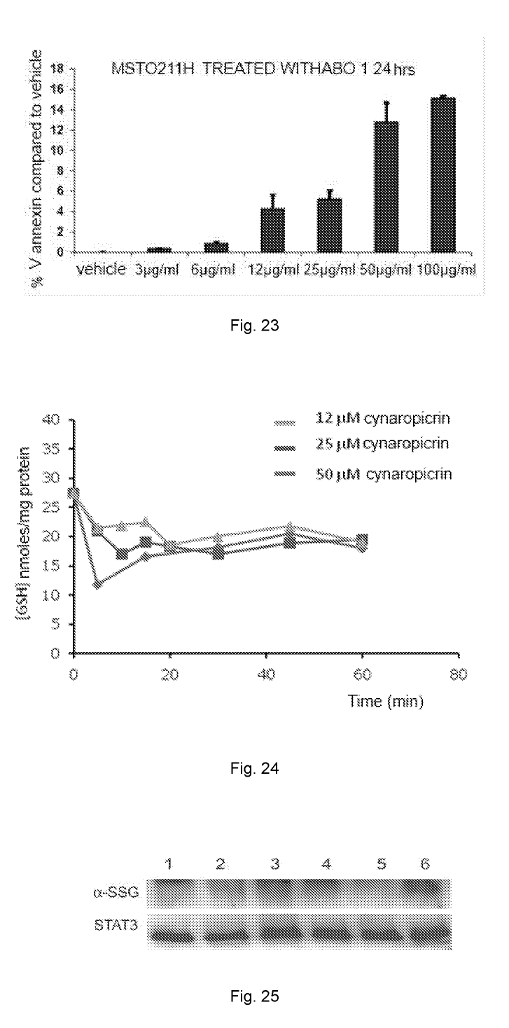

FIG. 23 assay to assess the induction of apoptosis by means of measurement of the level of annexin V. The extract of the invention induces apoptosis in the cell line MSTO211H, as determined by the coloration of annexin V, in a time-dependent and dose-dependent manner.

FIG. 24 Analyses of the intracellular concentration of GSH (see experimental section for the conditions) following treatment with various concentrations of cynaropicrin: triangles 12.5 μM, squares 25 μM, diamonds 50 μM.

Cynaropicrin determines a time-dependent and dose-dependent reduction of the intracellular concentration of GSH.

FIG. 25 Assay of glutathionylation of STAT3 (see experimental section for the conditions). Cynaropicrin determines the glutathionylation of STAT3. Lane 1. Control, Lane 2 GSH 1 mM, Lane 3 diamide 0.5 mM, Lane 4 GSSG, Lane 5 cynaropicrin 12 microM, Lane 6 cynaropicrin 25 μM.

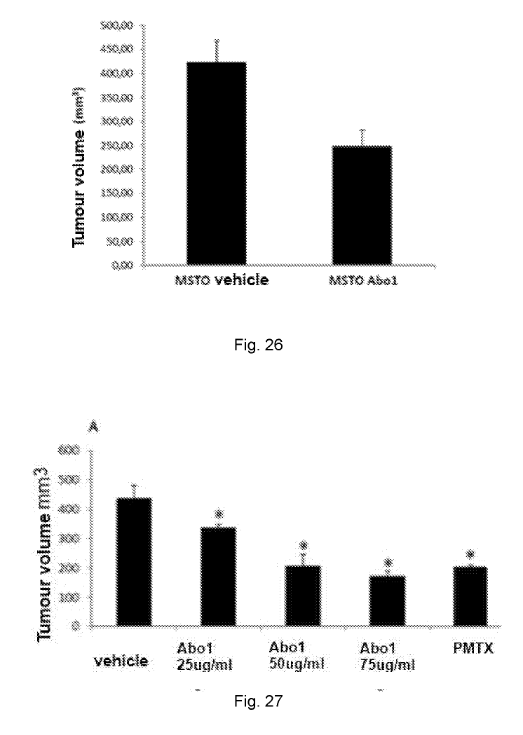

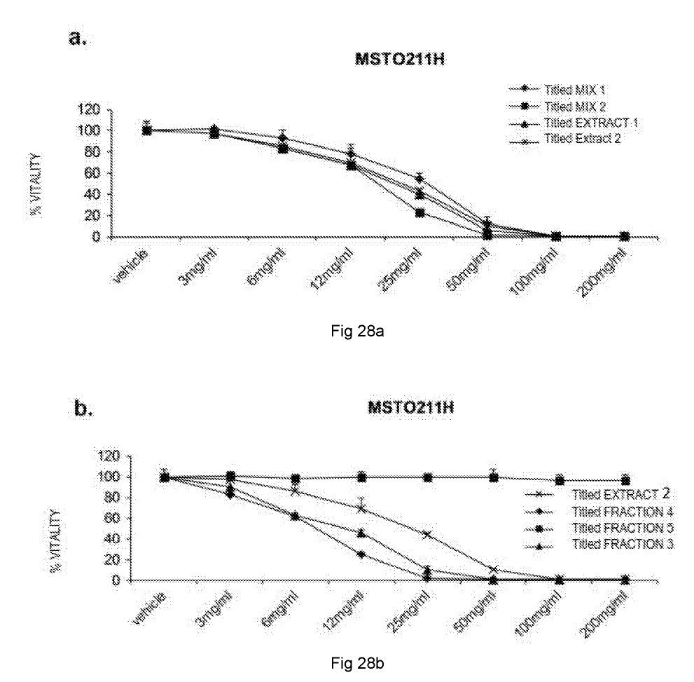

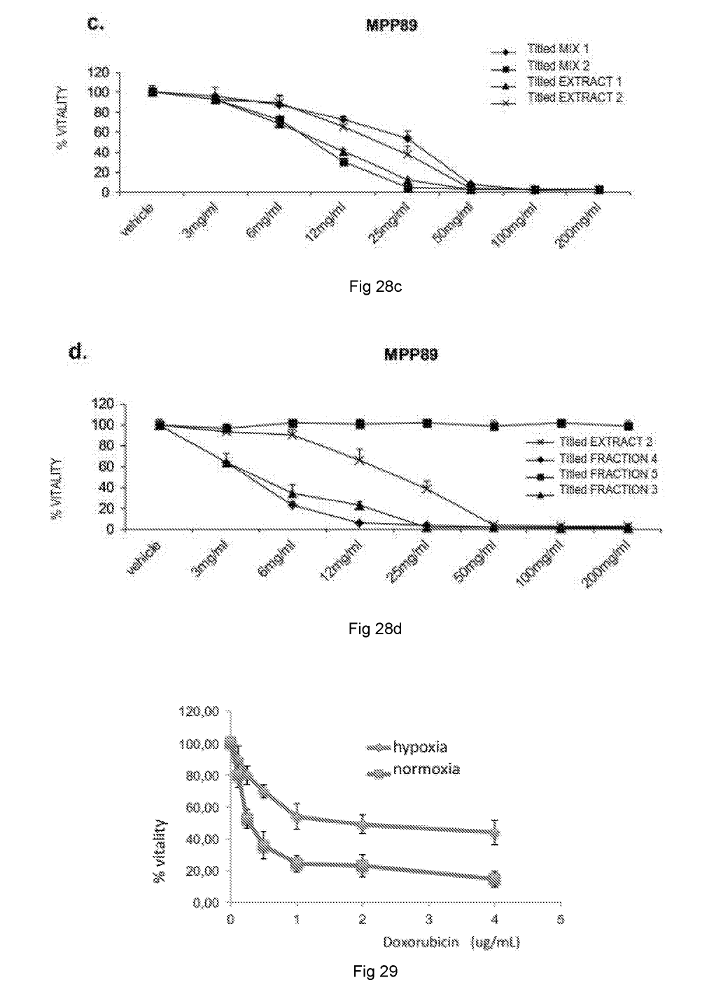

The data obtained in this experiment demonstrate that cynaropicrin lowers the intracellular concentration of GSH (FIG. 26) and that the variation of the redox state induces glutathionylation of STAT3, preventing the phosphorylation thereof (FIG. 27). The restoration of the physiological values of GSH, by means of pre-treatment with glutathione ethylene ester, reverses the ability of cynaropicrin to inhibit the phosphorylation of STAT3.

FIGS. 26 and 27 concern the assessment of the anti-tumour activity of the artichoke extract in the cell line MSTO211H, performed in nude female CD1 mice 6-7 weeks old (MPM tumour engraftment)

FIG. 26: Effect of artichoke on the engraftment of MPM cell lines

The MSTO211H were pre-treated with artichoke extract for 24 hours. Then, they were inoculated in nude CD1 mice. The pre-treatment with the artichoke extract influenced the engraftment of the tumour and induced a significant statistical difference (p=0.01) in the volume of the tumour.

FIG. 27: Effect of the artichoke extract on the transplantation of MPM cells. CD1 mice with xenograft of MSTO treated with growing quantities of artichoke extract for 3 weeks. A therapeutic dose-dependent effect was observed for the artichoke extract. Pemetrexed (PMTX) was used as positive control at a known therapeutic concentration. The figure shows the efficacy of the extract of the invention compared with the known therapeutic concentration of pemetrexed *p<0.01.

FIG. 28: Comparison of the three vitality curves with ATPlite assay on malignant pleural mesothelioma cell lines (FIGS. 28a and 28b MSTO211H, FIGS. 28c and 28d MMP-89).

In graphs a and c the assay was performed with various extracts of Cynara scolymus or with a mix of fractions of extract of Cynara scolymus, wherein total caffeoylquinic acids represent from 9% to 15% by weight of the extract or of the mixture (mix) of fractions in dry form, chlorogenic acid represents from 3.5% to 5.5% by weight of the extract or of the mixture of fractions in dry form, and cynaropicrin represents from 0.2% to 3% by weight of the extract or of the mixture of fractions in dry form.

In graphs b and d the assay was performed with a titrated extract reported also in graphs a and c, with the fractions 3, 4 and 5 as described below.

Apparently, fractions 3 and 4 are effective at least as much as an entire extract or mix titrated as described above, whereas fraction 5 alone has no effectiveness whatsoever.

FIG. 29: Treatment with MDA-MB231 cells, cultivated under normoxia and chronic hypoxia, treated for 24 hours with various concentrations of doxorubicin. Obtained data show that ChR-MDA-MB231 cells are more resistant to the drug with respect to parental ones cultivated under normoxia.

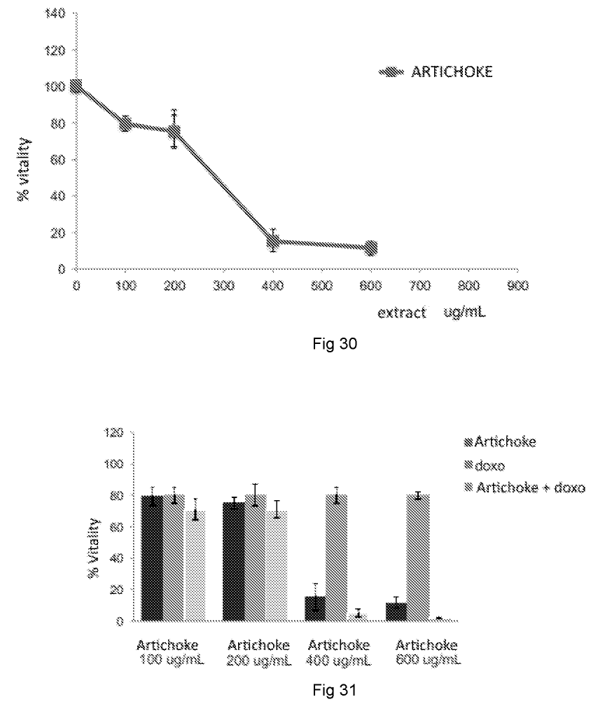

FIG. 30: Treatment with titrated extract according to the invention. MDA-MB231 cells, cultivated under normoxia and chronic hypoxia, were treated for 24 hours with various concentrations of titrated extract according to the invention, dissolved in 50% EtOH at the concentration of 100 mg/ml. The results obtained show that the extract inhibits cell vitality in a dose-dependent way (EC₅₀=300 ug/mL).

FIG. 31: Treatment with titrated extract according to the invention and doxorubicin. For the purpose of sensitising cells to chemotherapeutic treatments, ChR-MDA-MB231 cells were treated with increasing doses of the extract according to the invention in combination with 0.25 ug/mL doxorubicin, and cell vitality was assessed with Trypan blue as indicated in the experimental section below. For this type of experiments, an amount of doxorubicin able to induce a 20% mortality in examined cells was used.

### DETAILED DESCRIPTION OF THE INVENTION

The present application thus relates to an extract of Cynara scolymus or a fraction of extract of Cynara scolymus or a mixture of said extract with one or more of said fractions or a mixture of said fractions, titrated in total caffeoylquinic acids, in chlorogenic acid and in cynaropicrin, wherein
total caffeoylquinic acids represent from 8% to 16% by weight of said extract or of said fraction or of said mixture in dry form, chlorogenic acid represents from 3.5% to 7% by weight of said extract or of said fraction or of said mixture in dry form, and said cynaropicrin represents from 0.2% to 4% by weight of said extract or of said fraction or of said mixture in dry form,
   or wherein
total caffeoylquinic acids represent from 25% to 48% by weight of said fraction in dry form, chlorogenic acid represents from 11% to 21% by weight of said fraction in dry form, and said cynaropicrin represents from 1% to 10% by weight of said fraction in dry form
   in combination with one or more anti-tumour or anti-inflammatory drugs for use in the prevention and/or in the treatment of an inflammatory and/or pre-tumour and/or tumour pathological condition characterised by a constitutive or anomalous activation of the STAT3 transcription factor.

In a particular embodiment of what mentioned above, the present application relates to an extract of Cynara scolymus or a fraction of extract of Cynara scolymus or a mixture of said extract with one or more of said fractions or a mixture of said fractions, titrated in total caffeoylquinic acids, in chlorogenic acid and in cynaropicrin, wherein
total caffeoylquinic acids represent from 9% to 15% by weight of said extract or of said fraction or of said mixture in dry form, chlorogenic acid represents from 3.5% to 5.5% by weight of said extract or of said fraction or of said mixture in dry form, and said cynaropicrin represents from 0.2% to 3% by weight of said extract or of said fraction or of said mixture in dry form,
   or wherein
total caffeoylquinic acids represent from 25% to 35% by weight of said fraction in dry form, chlorogenic acid represents from 11% to 15% by weight of said fraction in dry form, and said cynaropicrin represents from 1% to 8% by weight of said fraction in dry form
   in combination with one or more anti-tumour or anti-inflammatory drugs for use in the prevention and/or in the treatment of an inflammatory and/or pre-tumour and/or tumour pathological condition characterised by a constitutive or anomalous activation of the STAT3 transcription factor.

As indicated before, the anomalous or constitutive activation would appear to consist in an anomalous or constitutive phosphorylation of this factor with resultant inflammatory and/or tumourigenic effects both in the blood and in tissues.

In the following description, in the claims and in the drawings, the term “STAT3” denotes the transduction factor of the signal and activation of STAT3 transcription (Signal Transducer and Activator of Transcription 3). Conventionally, where reference is made to the gene, uppercase italicised letters are used, whereas the protein is indicated by non-italicised uppercase letters.

It is already known in the literature that inflammation and tumours are closely linked by oncogenic and environmental pathways, and the phosphorylation of the STAT3 factor (Signal Transducer and Activator of Transcription 3) causes activation thereof and the displacement of the nucleus where it acts as an activator of the transcription of numerous cytokines, chemokines, and other mediators associated with inflammation, thus promoting cancer.

Inhibitors of the activation of STAT3 are therefore factors that have a preventative and/or curative effect towards all those pathologies in which constitutive activation of the STAT3 factor is present.

Artichoke or Cynara scolymus for the purposes of the present invention mean plants belonging to the Cynara (Cynara spp.) genus, in particular Cynara cardunculus subsp. scolymus.

For the purposes of the implementation of the present invention, the extract may be an extract of leaves and/or flower-heads or mixtures thereof, either fresh of dried.

The term “flower-heads” denotes the head of the flowers produced by the plant, for example the artichoke itself (part commonly used as food). The extract could be a fluid extract, or an extract lyophilised or dried by means of known drying techniques. The extract can be obtained by means of extraction with the following solvents: water, ethanol, methanol, acetone or isopropanol, in each case in pure form or in a mixture with one another. The alcohol could be methanol, ethanol, isopropanol and is preferably ethanol. The ethanol can be used in pure form or in mixture with water at the following percentages: 96%, 90%, 85%, 80%, 75%, 70%, 65%, 60%, 55%, 50%, 45%, 40%, 35%, 30%, 25%, 20%, 15%, 10%, 5%, 1%. In a non-limiting embodiment of the invention, the solvent used for the extraction could be a mixture formed by ethyl alcohol and water in a proportion of 50:50. The fluid extract could be prepared by means of hydroalcoholic extraction by percolation/digestion of the artichoke leaves in relation to drug/solvent from 1:2 to 1:100 and preferably in a ratio of 1:10. The duration of the extraction is a duration commonly used by a person skilled in the art and could be, for example, from a minimum of 1 hour to approximately 8 hours. The temperature of extraction is normally controlled and could preferably be, for example, a temperature of approximately 50° C. The evaporation of the alcohol from the hydroalcoholic extract and the subsequent drying of the aqueous concentrate could be performed by means of lyophilisation or desiccation to provide the lyophilised extract or dry extract.

The preparation of such extracts is commonly known to a person skilled in the art and does not need to be described in particular detail in the present disclosure. For the purposes of implementing the present invention, it is possible to use any extract among those indicated above, prepared in accordance with conventional techniques.

In particular, for the purposes of the present invention, the extract could also be substituted by a fraction or by a mixture of fractions of extract of Cynara scolymus, or by a mixture of extract of Cynara scolymus and of one or more fractions of extract as described above, as long as the above-disclosed titration criteria are met. Given the variability of plant extracts, which can result from climatic conditions, environmental conditions, from differences of cultivation grounds and/or cultivation techniques, or even by the different varieties of cultivated plants, for a clinical use it is important to standardize the product and to identify standardization parameters enabling to afford a product with defined features.

The authors of the present invention have therefore characterized the extracts used in the experimenting, such as, e.g., those reported in the figures and in the experimental protocols, in order to identify parameters enabling to standardize the end product to be used in clinical practice.

The extracts used were then titrated for some active components, and were also fractionated with various techniques in order to be able to obtain fractions of extract that were titrated and titratable for the same components and be therefore able to use also individual fractions, or to mix two or more of said fractions in order to obtain an end product falling within the parameters indicated above.

The titration of the parameters is performed on dry samples, e.g., dried, dehydrated or lyophilized (freeze dried).

According to the present invention, total caffeoylquinic acids represent from 8% to 16% or from 9 to 15% by weight of said extract or of said fraction or of said mixture in dry form, such as, e.g., about 8%, 9%, 10%, 11%, 12%, 13%, 14%, 15%, 16%. The indication “about” here means that also non-integers from 8 to 16, such as, e.g., 8.1; 8.2; 8.3; etc., up to 16, are encompassed by the invention.

In a preferred embodiment, total caffeoylquinic acids represent from 11% to 13% by weight of said extract or of said fraction or of said mixture in dry form, such as, e.g., about 11%, 12%, 13% and non-integers comprised from 11 to 13.

According to the present invention, the total chlorogenic acid represents from 3.5% to 7%, or from 3.5% to 5.5% or from 4.5% to 5.5% by weight of said extract or of said fraction or of said mixture in dry form, such as, e.g., about 4.5%, 4.6%, 4.7%, 4.8%, 4.9, 5.0%, 5.1%, 5.2%, 5.3%, 5.4%, 5.5%.

According to the present invention, total cynaropicrin represents from 0.2% to 4% or from 0.2 to 3% by weight of said extract or of said fraction or of said mixture in dry form. Since cynaropicrin tends to degrade, the initial cynaropicrin content (i.e., just as the extraction or the fractioning have occurred) is preferably ranging from 2 to 3%. In any case, it is acceptable that the extract, the fraction or the mixture of fractions reach a cynaropicrin content equal to at least 0.2% at +36 months from extraction, when stored at a temperature ranging from +4° C. to +40° C., preferably at 25° C., from the extraction or from the fractioning.

The present invention also relates to a fraction of extract of Cynara scolymus wherein the percentages (percents) by weight of each one of the components indicated above are about twice those reported above, and therefore a fraction titrated in total caffeoylquinic acids, in chlorogenic acid and in cynaropicrin, wherein total caffeoylquinic acids represent from 25% to 48% or from 25% to 35% by weight of said fraction in dry form, chlorogenic acid represents from 11% to 21% or from 11% to 15% by weight of said fraction in dry form and said cynaropicrin represents from 1% to 10% or from 1% to 8% by weight of said fraction in dry form.

This fraction is suitable for all uses and implementations as compositions and kit and therapeutic method indicated in the description for the extracts, the fractions and the mixtures of fractions titrated in total caffeoylquinic acids, in chlorogenic acid and in cynaropicrin, wherein total caffeoylquinic acids represent from 8% to 16% or from 9% to 15% by weight of said extract or of said fraction or of said mixture in dry form, chlorogenic acid represents from 3.5% to 7% or from 3.5% to 5.5% by weight of said extract or of said fraction or of said mixture in dry form and said cynaropicrin represents from 0.2% to 4% or from 0.2 to 3% by weight of said extract or of said fraction or of said mixture in dry form.

According to the present invention, numerous fractions of extract of Cynara scolymus can be obtained by various methods that are indicated below. The fractions, once obtained, are titrated in total caffeoylquinic acids, in chlorogenic acid and in cynaropicrin and can then be selected, thanks to the optimal titers disclosed in the present description, fractions that can be used alone or mixtures of fractions having a concentration by weight of each one of the three titrated components as defined above.

By way of example, various fractions can be obtained by following the various methods and steps listed below:

1. Dried leaves and/or flower-heads of Cynara scolymus are fractionated, put into contact with 96° ethyl alcohol for a period ranging from 4 to 10 hours at a temperature ranging from 35 to 45° C. The alcoholic part is separated from the leaves and/or flower-heads and is subjected to filtration so as to eliminate plant residues. The clarified alcoholic solution is collected.
2. The plant residue is subjected to a further extraction with water, preferably demineralized, and the aqueous component is collected, subjecting it also to filtration so as to remove residual plant parts.

The clarified aqueous solution is collected.

3. The collected clarified (alcoholic and aqueous) solutions are mixed, obtaining an alcoholic solution ranging from 40° to 60° (in the present invention, with respect to alcohols, the sign ° denotes alcoholic grades) and is subjected to precipitation and centrifuging, with recovery of the supernatant that is subjected to filtration.
4. The precipitate obtained after supernatant removal is collected and corresponds to a first fraction of extract that can then be dried, e.g. by freeze drying, and titrated. On average, in the first fraction obtained, total caffeoylquinic acids represent about 2-3% of the total weight of the dry fraction of extract, chlorogenic acid represents about 0.2-0.6% of the total weight of the dry fraction of extract, and cynaropicrin represents about 0.1-0.3% of the total weight of the dry fraction of extract.
5. The supernatant of the 40°-50° hydroalcoholic fraction subjected to precipitation and centrifuging as described above at point 3 is concentrated under vacuum, eliminating the alcohol and therefore bringing the fraction to alcoholic 0°, the obtained concentrated aqueous solution is subjected to precipitation and centrifuging and the supernatant is subjected to filtration for clarification.
6. The precipitate obtained after supernatant removal is collected, and it corresponds to a second fraction of extract that can then be dried, e.g. by freeze drying, and titrated.

On average, in the second fraction obtained, total caffeoylquinic acids represent about 1-2.5% of the total weight of the dry fraction of extract, chlorogenic acid represents about 0.05-0.1% of the total weight of the dry fraction of extract, and cynaropicrin represents about 0.3-0.5% of the total weight of the dry fraction of extract.

7. The filtrate obtained from the supernatant after the centrifuging and subjected to filtration at point 5 is an aqueous solution that is concentrated, e.g. under vacuum, and then dried, e.g. by freeze drying, and therefore corresponds to a third fraction. On average, in the third fraction obtained, total caffeoylquinic acids represent about 12-14% of the total weight of the dry fraction of extract, chlorogenic acid represents about 5-7% of the total weight of the dry fraction of extract, and cynaropicrin represents about 2.5-3.5% of the total weight of the dry fraction of extract.
8. Alternatively, the filtrate obtained from the supernatant after centrifuging and subjected to filtration at point 5 is an aqueous solution which is subjected to adsorption on high-porosity adsorbing resin.
9. The resin-adsorbed fraction is then recovered, concentrated and dried, e.g. by freeze drying, and corresponds to a fourth fraction.

On average, in the fourth fraction obtained, total caffeoylquinic acids represent about 29-32% of the total weight of the dry fraction of extract, chlorogenic acid represents about 13-15% of the total weight of the dry fraction of extract, and cynaropicrin represents about 3-4.5% of the total weight of the dry fraction of extract.

10. The fraction not adsorbed on resin is dried, e.g. by freeze drying, and corresponds to a fifth fraction.

On average, in the fourth fraction obtained, total caffeoylquinic acids represent about 0.5-0.7% of the total weight of the dry fraction of extract, chlorogenic acid represents about 0.1-0.2% of the total weight of the dry fraction of extract and cynaropicrin represents about 0.04-0.06% of the total weight of the dry fraction of extract.

According to the present invention, step 8 can be carried out on a column or on a bed with resins, able to adsorb aromatic substances or substances rich in highly unsaturated portions, or rich in alkyl or cycloalkyl groups, and to let elute non-related substances, such as many polar nonaromatic substances. The resin-adsorbed is desorbed with a suitable solvent, like, e.g., ethanol or hydroalcoholic solvents, for other uses.

Suitable chromatography resins may be, e.g., high-porosity adsorption resins of styrene-divinyl benzene copolymer, like, e.g., amberlite XAD-2, serdolit PAD-II, ADS TQ 318.

In particular, the resin will be a resin able to adsorb aromatic and/or apolar substances, like, e.g., a hydrophobic adsorbing resin, the resin consists of microspheres of a diameter of 0.2 mm 0.8 mm, with an uniformity coefficient 1.5 obtained by polymerization of Styrene and DVB without active groups, characterized by a highly porous physical structure having the parameter relative to the pore volume equal to about 1.3 ml/g enabling adsorption and selective elution of organic substances, preferably of aromatic nature.

The table below reports punctual titrations data obtained on an extract of Cynara scolymus used in the experimenting reported (ABO-1) and on its fractions obtained according to the methods reported above (therefore, first, second, third, fourth and fifth fraction).

| | | Chlorogenic | total caffeoylquinic |
|---|---|---|---|
| | Cynaropicrin | acid | acids |
| | % by weight | % by weight | % by weight |
| Freeze dried Cynara | 2.05 | 5.4 | 11.85 |
| Scolymus extract | | | |
| (ABO-1) | | | |
| l^{st} fraction | 0.24 | 0.44 | 2.48 |
| 2^{nd} fraction | 0.44 | 0.082 | 1.96 |
| 3^{rd} fraction | 3.14 | 5.89 | 13.37 |
| 4^{th} fraction | 6 | 32.01 | 19.68 |
| 5^{th} fraction | 0.05 | 0.61 | 0.82 |

By way of example, therefore, the mixture of fractions may be a mixture comprised of fraction 1 by about 12%, of fraction 2 by about 6%, and of fraction 3 by about 82%, or a mixture comprised of fraction 1 by about 12%, of fraction 2 by about 6%, of fraction 4 by about 55% and of fraction 5 by about 27%.

By titrating the obtained fractions, a person skilled in the art will certainly know how to reconstitute a mixture of fractions or to supplement an extract particularly poor in pharmaceutically active ingredients to obtain a compound with the optimal titration indicated above.

Fractions 3 and 4 are useful as such, as is apparent from data reported in FIG. 28. In the present invention the [Italian] term “active pharmaceutical ingredient” is equivalent to the English term “Active pharmaceutical ingredient” (API).

The term “active pharmaceutical ingredient” can also be replaced by the term “active ingredient” (or “active principle”) meant as set of molecules with pharmacological activity.

For the purposes of the present description, as “active pharmaceutical ingredient” of the invention” are meant:

a. an extract of Cynara scolymus or a fraction of extract of Cynara scolymus or a mixture of said extract with one or more of said fractions or a mixture of said fractions, titrated in total caffeoylquinic acids, in chlorogenic acid and in cynaropicrin, wherein total caffeoylquinic acids represent from 8% and 16% or from 9% to 15% by weight of said extract or of said fraction or of said mixture in dry form, chlorogenic acid represents from 3.5% to 7% or from 3.5% to 5.5% by weight of said extract or of said fraction or of said mixture in dry form, and said cynaropicrin represents from 0.2% to 4% or from 0.2 to 3% by weight of said extract or of said fraction or of said mixture in dry form (above-described detailed embodiments included);

b. a fraction of extract of Cynara scolymus titrated in total caffeoylquinic acids, in chlorogenic acid and in cynaropicrin, wherein total caffeoylquinic acids represent from 25% to 48% or from 25% to 35% by weight of said fraction in dry form, chlorogenic acid represents from 11% to 21% or from 11% to 15% by weight of said fraction in dry form, and said cynaropicrin represents from 1% to 10% or from % to 8% by weight of said fraction in dry form.

In accordance with the present invention, the extract of Cynara scolymus or a fraction thereof or a mixture of fractions thereof titrated in total caffeoylquinic acids, in chlorogenic acid, and in cynaropicrin, as described in detail above and in the claims, could be used as active pharmaceutical ingredient for the prevention and/or the treatment of diseases characterised by a constitutive or anomalous activation of the STAT3 transcription factor.

Such diseases can be, for example and as noted in the literature, diseases of the inflammatory and/or pre-tumour and/or tumour type.

For the purposes of the present invention, the pathological states characterised by a constitutive or anomalous activation of the STAT3 transcription factor can be caused by viral infections (as noted in the literature), such as infections by H. pylori, infections by the Hepatitis B virus, infections by HPV (human papilloma virus), infections by the Epstein-Barr virus (as reported in Yu et al 2009).

As already mentioned, the term “STAT3” thus denotes the human transcription factor “Signal transducer and activator of transcription 3”, coded in humans by the STAT3 gene.

The invention concerns pathological states in humans defined in detail in the present description (for example below) in which this gene is activated constitutively or anomalously in any case.

The pre-tumour pathological states in which a constitutive or anomalous activation of STAT3 is present can be either pathological states following the ablation of a tumour, and thus pre-tumour in the sense that the tumour could reform, or pathological states in which there is a transfer from inflammation to the acquisition of malignant characteristics on the part of the cell, as reported in the literature.

In accordance with the present invention, the tumour pathological states can be any tumours characterised by a constitutive or anomalous activation of STAT3 reported in the prior art, such as, e.g.:

prostate cancer, multiple myeloma, lymphoma, melanoma, carcinoma of the ovaries, breast cancer, carcinoma of the renal cells, pancreatic adenocarcinoma, lung cancer, brain tumour, erythroleukaemia, squamous-cell carcinoma of the head and neck, colon cancer, mesothelioma (which is intended to mean malignant pleural mesothelioma, or MPM).

More specifically, said brain tumour could be, for example, a glioma, a brain meningioma, a medulloblastoma, said lymphoma could be Sezary syndrome, EBV-associated Burkitt lymphoma, Samiri HSV-dependent lymphoma, cutaneous T-cell related lymphoma; said leukaemias may be HTLV-I-dependent leukaemia, chronic lymphocytic leukaemia (CLL), acute myelogenous leukaemia (AML), megakaryocytic leukaemia, large granular lymphocytic leukaemia (LGL).

In accordance with a non-limiting example of the present invention, the extract of Cynara spp. as defined above can be used for the prevention and/or the treatment of any one of the pathological states characterised by a constitutive or anomalous activation of STAT3 listed in Table 1 above.

The terms “constitutive activation” or “anomalous activation” according to the present invention are to be understood within the sense of the meaning attributed to such terms in the literature relating to STAT3 (for example as listed in the bibliography), or a persistent activation of this factor, usually absent in healthy cells. A specificity exists in the inhibition of the activation and of the activity of STAT3 shown by the active pharmaceutical ingredient of the invention in the treatment of tumour pathologies resistant to treatment with chemotherapeutic agents that do not inhibit STAT3. A non-limiting example of chemotherapeutic agents that do not inhibit STAT3 is represented by the chemotherapeutic drugs used for mesothelioma, which is a tumour with pSTAT3 constitutively activated and highly chemo-resistant. Examples of the agents commonly used are represented by pemetrexed, which is an inhibitor of thymidylate synthase; methotrexate, which is a competitive and reversible inhibitor of dihydrofolate reductase; gemcitabine, which inhibits the synthesis of DNA by acting as a false substrate in the biosynthetic pathways of the pyrimidine nucleotides; vinorelbine, which is an antimitotic drug that binds to the monomers of tubulin, inhibiting the formation of microtubules; cisplatinum, which is an agent able to interfere with all the phases of the cell cycle binding to the DNA by means of the formation of interfilament and intrafilament cross-links in the DNA.

The experimental data presented below and in the figures obtained on tumour cell lines in which the constitutive activation of STAT3 is known, also show that the extract of Cynara scolymus or a fraction thereof or a mixture of fractions thereof, titrated as disclosed in the present description, are associated advantageously with one or more anti-tumour drugs, thus increasing, also synergically, the anti-tumour efficacy of the drugs themselves.

Therefore, the extract of Cynara scolymus or a fraction thereof or a mixture of fractions thereof as described and claimed here will be used in the prevention and/or in the treatment of an inflammatory and/or pre-tumour and/or tumour pathological condition characterised by a constitutive or anomalous activation of the STAT3 transcription factor, in association with one or more compounds with anti-tumour activity and/or one or more compounds with anti-inflammatory action.

In accordance with an embodiment, the compound with anti-tumour activity can be a chemotherapeutic agent and can be selected from the group comprising cisplatinum, doxorubicin, pemetrexed, methotrexate, vinorelbine, gemcitabine and taxol.

The present invention also comprises the use of extract of an extract of Cynara scolymus or a fraction of extract of Cynara scolymus or a mixture of said extract with one or more of said fractions or of a mixture of said fractions titrated according to what disclosed in the present description in association with one or more chemotherapeutic agents for the prevention and/or the treatment of tumour or pre-tumour pathological states characterised by a constitutive or anomalous activation of STAT3.

In particular, the extract, the fraction or the mixture according to the present invention will be titrated in total caffeoylquinic acids, in chlorogenic acid and in cynaropicrin, wherein total caffeoylquinic acids represent from 8% to 16% or from 9% to 15% by weight of said extract or of said fraction or of said mixture (mix) in dry form, chlorogenic acid represents from 3.5% to 7% or from 3.5% to 5.5% by weight of said extract or of said fraction or of said mixture in dry form, and said cynaropicrin represents from 0.2% to 4% or from 0.2% to 3% by weight of said extract or of said fraction or of said mixture in dry form, or the fraction will be a fraction wherein total caffeoylquinic acids represent from 25% to 48% or from 25% to 35% by weight of said fraction in dry form, chlorogenic acid represents from 11% to 21% or from 11% to 15% by weight of said fraction in dry form, and said cynaropicrin represents from 1% to 10% or from 1% to 8% by weight of said fraction in dry form.

The detailed description of the titration ranges of total caffeoylquinic acids, chlorogenic acid and cynaropicrin provided above also applies to this specific embodiment.

The association with one or more chemotherapeutic agents may be a concomitant or sequential association, or the active pharmaceutical ingredient of the invention and the chemotherapeutic agents can be administered at the same time (in a single administration or in separate administrations) or over a period of time of a few minutes, or can be administered sequentially or at different times, separated from one another by more than a few minutes, over the course of the day or the period of therapeutic treatment.

The administration regime will be determined by the treating doctor in accordance with the sex, the age, the state of disease, the weight and the history of the patient. Both alone and in association, as described above, the treatment can be preventative, for example in cases of infection known to have possible tumourigenic effects such as those indicated above, or in the case of ablation of tumours so as to prevent said tumours from reforming.

The active pharmaceutical ingredient of the present invention can be formulated in compositions that can be used for the same objectives as described above.

The present invention therefore further relates to a composition comprising, as active pharmaceutical ingredient, an extract of Cynara scolymus, a fraction thereof or a mixture of fractions thereof or a mixture of said extract with one or more of said fractions thereof, titrated in total caffeoylquinic acids, in chlorogenic acid and in cynaropicrin as described above and as claimed, one or more anti-tumour agents and/or one or more anti-inflammatory agents, and a carrier and/or diluent and/or excipient for use in the prevention and/or in the treatment of an inflammatory and/or pre-tumour and/or tumour pathological condition characterised by a constitutive or anomalous activation of the STAT3 transcription factor.

The composition could, e.g., contain as sole active ingredients an extract of Cynara scolymus or a fraction of extract of Cynara scolymus or a mixture of said extract with one or more of said fractions or a mixture of said fractions, titrated in total caffeoylquinic acids, in chlorogenic acid and in cynaropicrin, wherein total caffeoylquinic acids represent from 8% to 16% or from 9% to 15% by weight of said extract or of said fraction or of said mixture in dry form, chlorogenic acid represents from 3.5% to 7% or from 3.5% to 5.5% by weight of said extract or of said fraction or of said mixture in dry form and said cynaropicrin represents from 0.2% to 4% or from 0.2% to 3% by weight of said extract or of said fraction or of said mixture in dry form, and one or more anti-tumour agents and/or one or more anti-inflammatory agents.

Alternatively, the composition could comprise as sole active ingredients a fraction of extract of Cynara scolymus wherein total caffeoylquinic acids represent from 25% to 48% or from 25% to 35% by weight of said fraction in dry form, chlorogenic acid represents from 11% to 21% or from 11% to 25% by weight of said fraction in dry form, and said cynaropicrin represents from 1% to 10% or from 1% to 8% by weight of said fraction in dry form and one or more anti-tumour agents and/or one or more anti-inflammatory agents.

Furthermore, the composition could comprise excipients suitable for the type of formulation selected.

A person skilled in the art will be able to readily identify the best formulations.

The composition may comprise the artichoke-derived active pharmaceutical ingredient of the invention as defined here, in a lyophilised, dry or fluid form.

As already indicated, the extract and the fractions thereof can be obtained by extraction of the leaves of artichoke or of the flower-heads of artichoke or of mixtures of the aforementioned parts, whether fresh or dried, according to the methods described above.

According to the present invention, the composition as defined above can be used for the prevention and/or the treatment of pathologies characterised by a constitutive or anomalous activation of the STAT3 transcription factor.

Such diseases can be, for example and as noted in the literature, inflammatory and/or pre-tumour and/or tumour diseases. The definition of the various pathological states for which the composition of the invention can be used is the same as that specified above in relation to the therapeutic use of the extract of the invention.

For the purposes of the present invention, the composition can treat pathological states characterised by a constitutive or anomalous activation of the STAT3 transcription factor as already defined above, tumour pathological states as already defined above characterised by a constitutive or anomalous activation of STAT3, and pre-tumour pathological states in which a constitutive or anomalous activation of STAT3 is present as already illustrated beforehand within the scope of the present description.

Merely by way of example, the composition could be made in form of hard gelatine capsule and contain from 30% to 60% of artichoke-derived active ingredient as defined above in lyophilised form and from 70% to 40% of suitable pharmacologically inert excipients (like, e.g., microcrystalline cellulose).

The capsule could be, e.g., a capsule having a final weight of 300-500 mg.

Since the active compound as described herein could be in a lyophilised, dried or fluid form, a person skilled in the art could readily make pharmaceutical compositions suitable for the selected use.

According to a non-limiting example, the present invention, the composition as defined here can be used for the prevention and/or the treatment of any one of the pathological states characterised by a constitutive or anomalous activation of STAT3 listed in Table 1 above.

Therefore, the invention further relates to a composition comprising, beside the active pharmaceutical ingredient of the invention, one or more compounds with anti-tumour activity (anti-tumour compounds) and/or compounds with anti-inflammatory activity (anti-inflammatory compounds) for use in the prevention and/or the treatment of a pathological condition of inflammatory and/or pre-tumour and/or tumour type characterised by a constitutive or anomalous activation of STAT3 transcription factor.

In accordance with an embodiment, such compounds with anti-tumour activity (anti-tumour compounds) may be chemotherapeutic agents selected, for example, from the group comprising cisplatinum, doxorubicin, pemetrexed, methotrexate, vinorelbine, gemcitabine and taxol.

Such drugs can be used with a standard dosage or with a dosage reduced with respect to that commonly used in chemotherapy.

The composition of the invention can be formulated in unit doses or in a dosable manner by the treating doctor for the purpose of also enabling therapies adapted to the individual needs of each patient.

The present invention thus envisages the use of compositions comprising as sole active pharmaceutical ingredients the active pharmaceutical ingredient of the invention, optionally in association with one or more further active ingredients having anti-tumour activity and/or one or more further active pharmaceutical ingredients having anti-inflammatory activity for the prevention and/or the treatment of tumour and/or inflammatory and/or pre-tumour pathological states characterised by a constitutive or anomalous activation of STAT3.

Such further active ingredients may be, for example, chemotherapeutic compounds, and the pathological states may be pre-tumour or tumour pathological states.

The association with one or more chemotherapeutic agents may be a concomitant or sequential association, or the active pharmaceutical ingredient of the invention and the chemotherapeutic agents can be administered at the same time (in a single administration or in separate administrations) or over a period of time of a few minutes, or can be administered sequentially or at different times, separated from one another by more than a few minutes, over the course of the day or the period of therapeutic treatment.

The administration regime will be determined by the treating doctor in accordance with the sex, the age, the state of disease, the weight and the history of the patient. The treatment described can be preventative, for example in cases of infections known to have possible tumourigenic effects such as those indicated above, or in the case of ablation of tumours so as to prevent said tumours from reforming.

In the composition as described above (consisting in the active pharmaceutical ingredient of the invention and at least one pharmaceutically acceptable excipient or adjuvant, optionally in association with one or more anti-tumour agents and/or one or more anti-inflammatory agents) at least one pharmaceutically acceptable excipient or adjuvant may be selected among excipients or adjuvants technically used in common pharmaceutical or cosmetic practice or in the food industry. The excipients used may belong to the category of diluents, solubilisers, disintegrators, binders, lubricants, surfactants, slip agents and anti-adherents.

If necessary, the composition may also contain flavourings, colorants and preservatives used commonly in the pharmaceutical, cosmetic and food industries.

The compositions can be in any formulation considered suitable by a person skilled in the art preparing formulations intended for oral administration (for example powders, granulates, capsules in hard or soft gelatine, tablets, syrups, drops, solutions and oral emulsions), inhalation (for example aerosols, liquid and powder sprays), topical administration (gels, ointments, emulsions, pastes, foams, anhydrous solid forms for topical application, and patches) and parenterally in accordance with the techniques currently used and known to a person skilled in the art (for example for subcutaneous use, intramuscular use, intravenous use or intradermal use). In all formulations, the use of technological excipients or adjuvants is determined by selecting the substances to be used on the basis of those used commonly in pharmaceutical practice.

In the preparation of formulations based on the active pharmaceutical ingredient in association or not with agents having anti-tumour activity, a person skilled in the art could use any of the excipients deemed useful in accordance with the prior art in order to obtain a stable preparation suitable for use in therapy. By way of example, in the category of diluents, it is possible to use diluents in solid formulations, such as sugars, polyalcohols (for example lactose, mannitol, sorbitol), cellulose, salts of inorganic acids (for example dibasic calcium phosphate), salts of organic acids including citrates, carbonate and bicarbonate titrates in the form of salts of sodium, potassium and calcium, or diluents in liquid forms, such as water, edible oils for oral use (sunflower oil, olive oil, corn oil, sweet almond oil, nut oil) or used in topical formulations (jojoba oil, short-chain, medium-chain or long-chain triglycerides), polyalcohols (glycerine, propylene glycols, hexylene glycol).

In the category of the disintegrators, it is possible to use, for example, natural or modified starches (corn starch, rice starch, potato starch), croscaramellose sodium, glycolate sodium starch, crospovidone; possible binders that can be used include natural products of the rubber type (guar gum, xanthan gum, gum arabic), sucrose and synthesis products, including polyvinyl pyrrolidone and semi-synthetic derivatives of cellulose.

The use of stearic acid and salts thereof, including the salt of magnesium, polymers of ethylene glycol, triglycerides and natural or synthetic waxes as lubricants has proven to be effective.

The surfactants are used to make one or more active ingredients contained in the formulations forming the basis of the invention more soluble or washable with water, these active ingredient acting alone or carried by one or more diluents. For example, sorbitan esters, sorbitan polyoxyethylene esters, sucrose esters and sodium lauryl sulphate can be cited.

The slip agents may be selected for example from colloidal silica, precipitated silica, whereas the anti-adherents that can be used include, for example, talc or starch.

In the preparation of injectable formulations, it is possible to choose those excipients that allow effective solubilisation or dispersion of the active substance(s). By way of example, together with water, other hydrosoluble carriers such as polyalcohols and salts of organic or inorganic acids can be used for the purpose of obtaining pH and osmolarity suitable for the administration by means of injections.

In particular cases, it will be possible to use non-hydrosoluble carriers, such as oils, or substances of synthesis commonly approved for injective use.

A person skilled in the art can prepare all the formulations using the common preparation schemas known to him.

Merely by way of example, a formulation in capsules can be prepared conveniently by grinding beforehand the active pharmaceutical ingredient of the invention, mixing in a common mixer the powder obtained together with one or more anti-tumour agents and the excipients selected to prepare the formulation, for example a diluent, a disintegrator, a lubricant and a slip agent selected from those mentioned above or available on the market and approved for oral use.

In the case of a tablet, it could be necessary to granulate some or all of the mixture with a binding agent dissolved beforehand in water or introduced in mixture and using the water as an adjuvant of the process of granulation in accordance with the prior art.

The granulate may be dried, sieved and further mixed with other powders for the purpose of obtaining a mixture suitable for obtaining tablets in accordance with that known to a person skilled in the art.

In the case of parenteral use, the composition may also be provided with the active ingredients in separate containers conveniently miscible in accordance with specific operational requirements.

For the purpose of facilitating the use of the compositions described here, these can be presented in the form of unit doses containing the active pharmaceutical ingredient of the invention and optionally one or more anti-tumour agents and/or one or more anti-inflammatory agents, effective for a preventative and/or therapeutic use of a particular pathological condition characterised by a constitutive or anomalous activation of the STAT3 transcription factor.

The present invention further relates to a kit for the concomitant or sequential administration of the active pharmaceutical ingredient of the invention and one or more compounds with anti-tumour activity and/or one or more compounds with anti-inflammatory activity for use in the prevention and/or in the treatment of an inflammatory and/or pre-tumour and/or tumour pathological condition characterised by a constitutive or anomalous activation of the STAT3 transcription factor, said kit comprising one or more aliquots of the active pharmaceutical ingredient of the invention as defined in the present description, and one or more aliquots of one or more compounds with anti-tumour activity and/or one or more aliquots of one or more compounds with anti-inflammatory activity.

Alternatively, the kit may comprise one or more aliquots of the composition containing, as sole active pharmaceutical ingredient, the active pharmaceutical ingredient of the invention as defined in the present description and one or more aliquots of one or more compounds with anti-tumour activity and/or one or more aliquots of one or more compounds with anti-inflammatory activity.

As described above, such compounds can be chemotherapeutic agents selected for example from the group comprising cisplatinum, doxorubicin, pemetrexed, methotrexate, vinorelbine, gemcitabine and taxol.

The pathologies that can be treated or prevented with the kit or with the composition of the present invention are those already indicated in the description above, pathological states characterised by a constitutive or anomalous activation of the STAT3 transcription factor that can be caused for example by viral infections (as noted in the literature), including infections by H. pylori, infections by the Hepatitis B virus, infections by HPV (human papilloma virus), infections by the Epstein-Barr virus (as reported in Yu et al 2009), or tumour pathological states that can be represented by any tumour characterised by a constitutive or anomalous activation of STAT3 reported in the prior art.

A non-limiting example of such tumours comprises:

prostate cancer, multiple myeloma, leukaemia, lymphoma, melanoma, carcinoma of the ovaries, breast cancer, renal cell carcinoma, pancreatic adenocarcinoma, lung cancer, brain cancer, erythroleukaemia, squamous-cell carcinoma of the head and neck, colon cancer, mesothelioma.

More specifically, said brain tumour could be, for example, a glioma, a brain meningioma, a medulloblastoma, said lymphoma could be Sezary syndrome, EBV-associated Burkitt lymphoma, Samiri HSV-dependent lymphoma, cutaneous T-cell related lymphoma; said leukaemias may be HTLV-I-dependent leukaemia, chronic lymphocytic leukaemia (CLL), acute myelogenous leukaemia (AML), megakaryocytic leukaemia, large granular lymphocytic leukaemia (LGL).

The pre-tumour pathological states in which a constitutive or anomalous activation of STAT3 is present can be either pathological states following the ablation of a tumour, and thus pre-tumour in the sense that the tumour could reform, or pathological states in which there is a transfer from inflammation to the acquisition of malignant characteristics on the part of the cell, as reported in the literature.

Lastly, the present description also concerns a therapeutic method for the prevention and/or the treatment of an inflammatory and/or pre-tumour and/or tumour pathological condition characterised by a constitutive or anomalous activation of the STAT3 transcription factor, comprising the step of administering to an individual in need of it a therapeutically active quantity of active pharmaceutical ingredient of the invention or of a pharmaceutical composition comprising as sole active pharmaceutical ingredients the active pharmaceutical ingredient of the invention, optionally in association with one or more anti-tumour and/or anti-inflammatory compounds.

The method forming the basis of the present invention can be carried out by administering to a subject who presents an inflammatory and/or pre-tumour and/or tumour pathological condition characterised by a constitutive or anomalous activation of the STAT3 transcription factor, therapeutically effective doses of the active pharmaceutical ingredient as defined here, optionally in association with one or more anti-tumour or anti-inflammatory drugs; or by administering therapeutically effective doses of the composition as defined here, optionally further comprising one or more anti-tumour and/or anti-inflammatory drugs, or by administering the extract and one or more anti-tumour and/or anti-inflammatory drugs using the kit as defined here.

The administration, as described above, can be performed concomitantly or sequentially in accordance with the administration regime selected by the doctor.

Numerous experimental data have been reported that demonstrate the efficacy of the extract according to the present invention.

In a specific embodiment of the invention, mesothelioma (in particular, malignant pleural mesotheliona) is excluded from the pathologies according to the invention.

### Used Cell Lines

L428 Human lymphoma cell line. Available from DSMZ ACC197
KARPAS-299 Human lymphoma cell line with constitutively activated STAT3.

Available from Cell Bank Australia #6072604

Human T-cell lymphoma cell line, established from peripheral blood of a human of 25 years of age with non-Hodgkin T-cell lymphoma cells in 1986, now classified as lymphoblastoid lymphoma cell line. Karpas 299 expresses Stat3 phosphorylated in tyrosine 705 and serine 727.
MSTO211H Human lung biphase mesothelioma cell line with constitutively activated STAT3. Available from ATCC #CLR-2081.

Human mesothelioma cell line, established from the pleural spill of a human of 62 years of age with mesothelioma (biphase malignant) who had not had any prior therapy. Cell line MSTO211H expresses high levels of pStat3). (Tsao et al. Inhibition of c-Src expression and activation in malignant pleural mesothelioma tissues leads to apoptosis, cell cycle arrest, and decreased migration and invasion. Mol Cancer Ther 2007; 6:1962-1972.)
DU145 Human carcinoma cell line available from ATCC #HTB-81

The cell line DU145 is a human prostate cancel cell line of moderate metastatic potential compared with PC3 cells, which have high metastatic potential. The DU145 cells are not hormone-sensitive and do not express PSA (prostate-specific antigen). The cell line DU145 expresses pStat3 in a constitutive manner.
HCT116 Human colon cancer cell line. Available from ATCC #CCL-247.
MDA-MB-231 Human mammary adenocarcinoma cell line. Available from ATCC #HTB-26. Breast cancer cell line MDA-MB-231, established from a patient in 1973, presents epithelial-like morphology and, phenotypically, presents fusiform cells. In vitro, cell line MDA-MB-231 expresses high levels of pSTAT3 (Berisha J et al. Stat3 is tyrosine-phosphorylated through the interleukin-6/glycoprotein 130/Janus kinase pathway in breast cancer. Breast Cancer Research 2007, 9:R32).
NCI-H2052 Human mesothelioma cell line. This cell line expresses pSTAT3 (Tsao et al. Inhibition of c-Src expression and activation in malignant pleural mesothelioma tissues leads to apoptosis, cell cycle arrest, and decreased migration and invasion. MolCancerTher 2007; 6:1962-1972.) Available from ATCC #CLR-5915
NCI-h28 Human stage-4 mesothelioma cell line. Available from ATCC#CRL-5820
MPP-89 Human mesothelioma cell line. Available from CABRI, access number ICLC HTL00012

The following examples show how the extract of Cynara scolymus of the present invention is able to:

reduce the vitality in mesothelioma cells (MSTO211H, MPP-89, NCI-H2052, NCI-H28) in a dose-dependent manner, acting less strongly on non-transformed mesothelial cells (HMC);

reduce the ability to form colonies in assays of clonogenic survival over the same cell lines,

induce cell death of malignant mesothelioma cells MM in apoptotic assays;

inhibit the migration and the proliferation of MM cells in wound healing assays;

sensitise the MM cells with successive treatments with a chemotherapeutic agent, such as pemetrexed;

induce damage in the DNA of MM cells whilst not inducing damage to the DNA of HMC cells;

reduce the ability of tumour transplantation with MSTO cells on cells pre-treated with the extract;

have a dose-dependent effect in the treatment of xenotransplantation of MSTO.

### EXAMPLES

### 1. Analysis of the Phosphorylation of STAT3 by Means of Western Blot.

Results Reported in FIGS. 1-3.

### 1.1. Cell Lysis and Western Blotting.

The cells were lysed in ice for 30 min in lysis buffer NP40 (50 mM Tris-HCl pH 7.4, 150 mM NaCl, 1% NP-40, 1 mM EGTA, 1 mM EDTA) complemented with inhibitors of protease and phosphatase (5 mM PMSF, 3 mM NaF, 1 mM DTT, 1 mM NaVO4). Equal amounts of total extracts of protein (30 μg) were broken down by means of denaturing electrophoresis (SDS-PAGE) in 8% polyacrylamide gel and transferred for 2 hours on nitrocellulose membrane. The membranes were blocked with a 5% solution of milk dissolved in TBS-Tween_20 0.05% for 1 hour and incubated with the specific primary antibodies. The following primary antibodies were used: anti-beta actin (A-2228, SIGMA), anti-pSTAT3 (Tyr-705) (sc8059, Santa Cruz) and anti-STAT3 (sc7179, Santa Cruz). The secondary antibodies were peroxidase-conjugated (Santa Cruz), and ECL reagents (Amersham, GE Healthcare, Piscataway, N.J., USA) were used for the chemiluminescence.

1.2. Treatment of the Cell Lines of MPMs and of Normal Commercial Mesothelial Cells (HMC) with Extract of Cynara Scolymus.

The cell lines of MPMs (MSTO-211H, NCI-H28, NCI-H2052, MPP89) were acquired from ATCC (Rockville, Md.) whilst the HMCs (Human Mesothelial Cells) were acquired from Tebu-Bio (France). All the lines were grown in monolayers at 37° C. and at 5% of CO2 in specific culture media. The artichoke extract was dissolved conveniently in a solution of water for injectable solutions and ethanol in a ratio of 1:1 at an initial concentration of 30 mg/ml. To test the anti-tumour property, the product was then added directly in the medium of the various cell lines using various concentrations and various times, as shown in the drawings.

1.3. Results

The results, shown in FIGS. 1 to 2, show how the assayed extract inhibits the phosphorylation of STAT3 compared with the controls not treated with the extract.

FIGS. 1 and 2 show the data obtained on MSTO211H cells treated with extract of Cynara scolymus in accordance with the description.

FIG. 1 shows the data with the control treated with just the carrier and extract of Cynara scolymus, 100 μg/ml of culture medium for 24 hours (actin control), and FIG. 2 shows the data with cells treated for 24 hours with various concentrations of extract of Cynara scolymus: 25 μg/ml, 50 μg/ml, 75 μg/ml.

As for FIG. 1, the data with the control treated with just the carrier and extract of Cynara scolymus are shown, 100 μg/ml of culture medium for 24 hours (actin control).

2. The Extract of Cynara scolymus and Cynaropicrin Inhibit Constitutive Activation of STAT3 in DU145 Cells and in KARPAS Cells:

As can be seen in FIGS. 17-20, both the extract of Cynara scolymus and cynaropricrin act on STAT3 in DU145 cells and in KARPAS cells. 200 μg/ml of extract that contains 0.181% of cynaropricrin contain 1.2 μM of cynaropricrin. The figures show that the effect observed with 25 μM of cynaropricrin is equal to the effect observed with 200 μg/ml of extract, with titre of cynaropricrin equal to 0.181%, that is to say comprising 1.2 μM of cynaropricrin. Since the dose of extract used contains 1.2 mM of cynaropricrin, the data obtained show that the extract is more effective than cynaropricrin.

### 3. Assay of Clonogenicity on Cells of Malignant Pleural Mesothelioma (MPM)

MPM cells (MSTO211H, NCI-H28; MPP-89; NCI-H2052) were seeded at 200 cells per well and were treated with various growing concentrations (control just with carrier; 12.5 μg/ml; 25 μg/ml; 50 μg/ml; 100 μg/ml, 200 μg/ml) of extract of Cynara scolymus in accordance with the present description. Each point was plated in duplicate in the 6-well multiwell. The colonies formed were stained with violet crystal 15-21 days later. The colony formation assay, also known as a clonogenic assay, is a technique used to assess the efficacy of anti-tumour compounds in terms of the ability of the tumour cells to form colonies from a single cell. A colony is considered to be a group of 50 or more cells (clones) originating from a single cell.

The results of the experiment, shown in FIGS. 3a-3d, show the dose-dependent ability of the extract of the invention to inhibit, in a dose-dependent manner, the formation of colonies in all the MPM cell lines assayed.

The same assay was also performed on HCT116 colon cancer cells, DU145 prostate cancer cells and MDA-MB-231 breast cancer cells. In this case too, the data shown in FIGS. 4a, b, e and c show the efficacy of inhibiting, in a dose-dependent manner, the formation of colonies from the extract of the invention.

### 4. ATPlite™ Cell Vitality Assay

The vitality of various cell lines following exposure to the extract of the invention at various concentrations was assessed using the ATPlite™ assay (Perkin Elmer) in accordance with the producer's instructions. Where indicated, the term “carrier” refers to a solution of water for injectable solutions and ethanol at a concentration of 1:1 used in the same volumes used for the treatments.

ATPLite™ is a system for monitoring the levels of adenosine triphosphate (ATP) based on the activity of firefly (Photinus pyralis) luciferase. This luminescence assay is an alternative to colorimetric, fluorometric and radioisotopic tests for the quantitative evaluation of the proliferation of cultured mammalian cells subjected to treatment with possible substances contained in the culture medium. The monitoring of ATP is used in fact to evaluate the cytostatic and anti-proliferative effects of a vast range of drugs, modifiers of the biological response, and biological compounds. The ATPLite™ assay system is based on the production of light caused by the reaction with addition of ATP luciferases and D-luciferin. The light emitted is proportional to the concentration of ATP within certain limits. The quantity of ATP in cells correlates with the cell vitality.

The cell vitality of various types of MPM cell lines (MSTO211H, MPP89, NCI-H28) and of HMC cells (untransformed mesothelial cells provided by willing donors) were assayed following treatment with various concentrations of extract according to the invention (control just with carrier; 12.5 μg/ml; 25 μg/ml; 50 μg/ml; 100 μg/ml, 200 μg/ml).

The graph in FIG. 5, which shows the results of the assay, shows that the extract is able to significantly reduce cell vitality in a dose-dependent manner.

The effects on cell vitality were also assayed on untransformed mesothelial cells (HMCs), towards which the extract forming the basis of the invention demonstrated lower cytotoxicity compared with the tumour lines (FIGS. 6A-6C).

### 5. WST Assay of Cell Vitality and Proliferation, Comparison Between the Cytotoxic Efficacy of the Extract of the Invention and Cynaropicrin.

Cytotoxicity was assayed using the WST assay (WSTs=water soluble tetrazolium salts) which utilises the ability of mitochondrial dehydrogenases to separate the tetrazole ring from the yellow-coloured WST molecule (tetrazolium salt) to give an orange formazan salt. The amount of formazan produced following the treatment of the cells with the substances being tested is measured using spectrophotometry and is proportional to the number of living cells. WST-1 and in particular WST-8 (2-(2-methoxy-4-nitrophenyl)-3-(4-nitrophenyl)-5-(2,4-disulfophenyl)-2H-tetrazolium) are advantageous with respect to MTT because they reduce outside the cells, in combination with PMS as electron mediator, to produce water-soluble formazan. Lastly, the WST assays: (1) can be read directly (in contrast with MTT, which requires a solubilisation phase), (2) and give a more effective signal than MTT, and (3) reduce the toxicity for the cells (in contrast with MTT, which produces insoluble formazan that accumulates within the cells).

The following WST assays were performed:

5.1 WST-1 assay on cell line DU145 with 50, 100 e 200 μg/ml of extract of Cynara scolymus at 24-48-72 hours, shown in FIG. 9

5.2 WST-1 assay on cell line DU145 at 24 and 48 hours with 0-100-200-300-400-500-600 μg/ml of extract of Cynara scolymus (cynaropicrin=1.361%), which inhibits, in a time-dependent and dose-dependent manner, the vitality of the DU145 cells. FIG. 7, which shows the assay, also shows the content in cynaropicrin, expressed both in μg/ml and in μM, of the assayed extract at concentrations 100 μg/ml; 200 μg/ml; 300 μg/ml; 400 μg/ml; 500 μg/ml; 600 μg/ml (comprising, respectively, 0.47 μM; 0.94 μM; 1.41 μM; 1.88 μM; 2.35 μM and 2.82 μM of cynaropicrin).

5.3 WST-1 assay on cell line DU145 at 24 and 48 hours with cynaropicrin 0-10-20-30-40-50-60 μM inhibits, in a dose-dependent and time-dependent manner, the vitality of DU145 cells. The results are shown in FIG. 8.

It would appear, by comparing FIGS. 7 and 8, that the assayed extract is more than 40 times more effective than cynaropicrin.

6. Assays of Cell Vitality in Co-Treatment with Chemotherapeutic Agents

Cell lines MSTO211H and NCI-H2052 were used to evaluate the effects of the association of extract of Cynara scolymus+anti-tumour drug.

The assay shown in FIG. 10 was performed using ATPlite™ assay (Perkin Elmer) in accordance with the producer's instructions.

A solution of water for injectable solutions and ethanol at a concentration of 1:1 was used in the same volumes used for the treatments.

Reagents:

pemetrexed (Alimta, Lilly) diluted in accordance with the producer's instructions.

6.1 Association of Extract of Cynara Spp. and Pemetrexed with ATPlite™ Assay

FIG. 10 shows the vitality curve for MSTO211H after 72 h of treatment with pemetrexed and pemetrexed in association with extract of Cynara spp.; Graph A shows the treatment with the extract at non-cytotoxic dose (6 μg/ml) and pemetrexed for the MSTO211H cells, whereas graph B shows the treatment with the extract at non-cytotoxic dose (6 μg/ml) and with pemetrexed (various concentrations) for NCI-H2052 cells, and graph C shows the treatment with the extract at non-cytotoxic dose (6 μg/ml) and with pemetrexed (various concentrations) for untransformed HMC cells. The concentrations of the assayed compound are plotted on the abscissa, whereas the cell vitality expressed in percentage is plotted on the ordinate.

FIGS. 10A and B show how the treatment with extract sensitises the tumour lines to the treatment with pemetrexed. In the curve with double treatment, it is clear how just a concentration of pemetrexed of 10 μM is sufficient to lower the cell vitality of the assayed lines. It is interesting to note that, in the non-tumour line, the extract has a protective effect towards pemetrexed.

6.2 Evaluation of Cell Vitality with WST-1 Assay

The assays were carried out in parallel with variable doses of cynaropicrin in place of the extract, to compare the efficacy of the extract and that of cynaropicrin.

FIG. 11 shows the data obtained by incubating DU145 cells with cisplatinum (graph A), doxorubicin (graph B) and taxol (graph C) with just the carrier (cntr), with two different concentrations of extract of Cynara scolymus (abo-1) with just drug and with two different concentrations of extract of Cynara scolymus (abo-1) in association with the drug.

The extract used in the experiments shown in FIG. 11 had a content of 0.181% in cynaropicrin. The figure thus shows the concentrations of cynaropicrin with 100 and 200 μg/ml of extract, equal respectively to 0.18 and 0.36 μg/ml of cynaropicrin. FIG. 12 shows the association between growing concentrations of extract of Cynara Scolymus (cynaropicrin=1.361%) and cisplatinum at fixed concentration of 15 μg/ml and FIG. 13 shows the association between extract of Cynara Scolymus (cynaropicrin=1.361%) and doxorubicin at fixed concentration of 2 μg/ml. The figure also shows the values for the treatment with just extract (black) or just drug (white).

FIGS. 14 and 15, similarly to FIGS. 12 and 13, show the results of the same experiments performed with cynaropicrin in place of the extract of the invention, and show how the extract is significantly more effective than cynaropicrin.

FIG. 14 thus shows the association between cynaropicrin at growing concentrations and cisplatinum at a fixed concentration of 15 μg/ml, and FIG. 15 shows the association between cynaropicrin and doxorubicin at a fixed concentration of 2 μg/ml.

The figure also shows the values for the treatment with just cynaropicrin (black) and just drug (white).

### 7. Wound Healing Assay

The wound healing assay (FIG. 16a-b) is simple, inexpensive, and one of the first methods developed for studying directional cell migration in vitro. This method mimics cell migration during would healing in vivo. The basic steps involve creating a “wound” in a cell monolayer, then monitoring a specific zone of the “wound” by capturing images at the beginning and at regular intervals during the cell migration necessary to close the “wound”. The MSTO211H cells cultivated with a confluency of 95% were seeded in 6-well plates and the “wound” (or cut) was made with a puncture by 10-microlitre sterile pipette to remove the cells. Digital micrographs were produced after the wounds at the indicated times. The final bar chart shows the efficacy of closure of the cut (quantification number of the cells in %) treated with carrier or ABO 1 at the indicated times.

### 8. Assay to Assess the Induction of Apoptosis

See FIGS. 22-23)

8.1 Western Blotting

The same technique as described in point 1.1 was used, and the following primary antibodies were used: anti-beta actin (A-2228, SIGMA), anti-caspase-3 (31A1067, Alexis), anti-caspase-7 (#9492, Cell Signalling) and anti-PARP (#9542S, Cell Signalling).

8.2 FACS Analysis and PI Staining and PI/Annexin V Staining Analyses

For the purpose of determining the effect of the extract of the invention on the cell cycle, a FACS analysis was performed.

For staining with propidium iodide (PI), the cells were seeded in 6-well plates at a density of 10⁴ cells/ml. After 24 h, the tumour cells were treated with indicated concentrations of the extract of the invention for various time intervals. The cells were collected in suspension and the adhered cells were washed in PBS, fixed with frozen ethanol (70% v/v) and stored at −20° C. For the analyses, the cells were washed in PBS 1× and suspended in a solution of PBS 1Z, PI (25 mg/ml) and RNase A (200 mg/ml).

For the PI/annexin V double staining, the treated cells were collected and resuspended in binding buffer (HEPES pH 7.4, CaCl2 2.5 mM, NaCl 140 mM). Aliquots of cells were incubated for 15 min with annexin V FITC and PI (5 mg/mL) (Invitrogen).

During all the FACS analyses, 10⁵ events were analysed for each sample. The flow cytometry analyses were performed on a GuavaEasyCyte 8HT (Millipore) flow cytometer.

As can be seen in FIG. 23, the extract of the invention induces apoptosis in MSTO211H cells, as determined by the annexin V staining, in a time-dependent and dose-dependent manner.

### 9. Assay on Glutathione

The variation of the cell redox state, caused by the variation of the ratio between reduced and oxidised glutathione, determines the glutathionylation of STAT3, preventing the phosphorylation thereof in tyrosine and consequently the activation thereof (Butturini E et al. PLoSOne. 2011; 6(5):e20174.).

9.1 Intracellular Analyses of GSH.

The intracellular concentration of GSH was evaluated by means of a colorimetric method. The cell extract, deproteinised by means of 10% trichloroacetic acid, was treated with dithio nitrobenzene (DTNB), and the quantity of TNB, which is released following the reaction with GSH, was evaluated by analysing the absorbance at 412 nm.

9.2 Glutathionylation of STAT3

STAT3 was immunoprecipitated by incubating the protein cell extract overnight with an anti-STAT3 antibody. The proteins obtained were separated by means of SDS-PAGE in non-reducing conditions and were transferred on PVDF membrane. The glutathionylated STAT3 was recognised using an anti-GSH antibody.

The data shown in FIGS. 24 and 25 demonstrate that cynaropicrin lowers the intracellular concentration of GSH (FIG. 24) and that the variation of the redox state induces the glutathionylation of STAT3, preventing the phosphorylation thereof (FIG. 25). The restoration of the physiological values of GSH, by means of pre-treatment with glutathione ethylene ester, reverses the ability of cynaropicrin to inhibit the phosphorylation of STAT3.

10. Transplantation of Tumour Cells Treated or Untreated with the Extract of the Invention

Description of the First Engraftment Experiment.

The MSTO211H cells were treated with artichoke at the concentration of 50 μg/ml for 24 hours. A suspension of 2×10⁶ of cells in PBS/Matrigel (BD Biosciences) was collected and inoculated in the right hip of nude female mice 4 weeks old. The volume of the tumours was monitored twice a week up to the 21^{st} day. The mice were sacrificed and the masses removed.

11. Transplantation of Tumour Cells in Mice and Treatment with Cynara Scolymus and Pemetrexed (FIG. 27)

Description of the Second Engraftment Experiment.

The cells were expanded prior to the implantation and were evaluated in terms of their vitality and contamination, that is to say were counted and resuspended in PBS at a concentration of 20×10⁶/ml. Matrigel was added to the suspension to obtain a final concentration of 10×10⁶ cells/ml of PBS Matrigel 1/1. The MSTO cells were inoculated under the skin in 48 mice.

When the tumour reached an average volume of 60 mm³, the mice were divided into 8 groups formed by 6 animals per group receiving different treatments.

Two groups received artichoke in drinking water for 7 days of the week during a period of three weeks; the other groups received pemetrexed intraperitoneally for 5 days of the week during a period of 3 weeks.

The groups have been outlined in this way in Table 5 below:

| | no. | | | | | | | | | | | | |
|---|---|---|---|---|---|---|---|---|---|---|---|---|---|
| | ani- | cell | no. | path- | | treatm. | start of | administrat. | treatm. | treatm. | start of | administrat. | treatm. |
| | mals | line | cells | way | volume | A | treatm. | method | regime | B | treatm. | method | regime |
| Group | 6 | MSTO | 2 × 10⁶ | SC | 0.2 | | | | | Cynara | after | OS | drinkin [symbol] |
| 1 | | | | | (matrigel) | | | | | extract | tumour | | water |
| | | | | | | | | | | 20 μg/ml | appear- | | |
| | | | | | | | | | | | ance | | |
| Group | 6 | MSTO | 2 × 10⁶ | SC | 0.2 | | | | | Cynara | after | OS | drinkin [symbol] |
| 2 | | | | | (matrigel) | | | | | extract | tumour | | water |
| | | | | | | | | | | 50 μg/ml | appear- | | |
| | | | | | | | | | | | ance | | |
| Group | 6 | MSTO | 2 × 10⁶ | SC | 0.2 | | | | | Cynara | after | OS | drinking |
| 3 | | | | | (matrigel) | | | | | extract | tumour | | water |
| | | | | | | | | | | 75 μg/ml | appear- | | |
| | | | | | | | | | | | ance | | |
| Group | 6 | MSTO | 2 × 10⁶ | SC | 0.2 | | | | | | | | |
| 4 | | | | | (matrigel) | | | | | | | | |
| Group | 6 | MSTO | 2 × 10⁶ | SC | 0.2 | Peme- | after | IP | 5 days in | Cynara | after | OS | drinking |
| 5 | | | | | (matrigel) | trexed | tumour | | succes- | extract | tumour | | water |
| | | | | | | (100 | appear- | | sion | 20 μg/ml | appear- | | |
| | | | | | | mg/kg) | ance | | | | ance | | |
| Group | 6 | MSTO | 2 × 10⁶ | SC | 0.2 | Peme- | after | IP | 5 days in | Cynara | after | OS | drinking |
| 6 | | | | | (matrigel) | trexed | tumour | | succes- | extract | tumour | | water |
| | | | | | | (100 | appear- | | sion | 50 μg/ml | appear- | | |
| | | | | | | mg/kg) | ance | | | | ance | | |
| Group | 6 | MSTO | 2 × 10⁶ | SC | 0.2 | Peme- | after | IP | 5 days in | Cynara | after | OS | drinking |
| 7 | | | | | (matrigel) | trexed | tumour | | succes- | extract | tumour | | water |
| | | | | | | (100 | appear- | | sion | 75 μg/ml | appear- | | |
| | | | | | | mg/kg) | ance | | | | ance | | |
| Group | 6 | MSTO | 2 × 10⁶ | SC | 0.2 | Peme- | after | IP | 5 days in | | | | |
| 8 | | | | | (matrigel) | trexed | tumour | | succes- | | | | |
| | | | | | | (100 | appear- | | sion | | | | |
| | | | | | | mg/kg) | ance | | | | | | |

SC = subcutaneous
treatm. = treatment
administrat. = administration
IP = intraperitoneal
OS = oral [symbol]
indicates data missing or illegible when filed

With appearance of progression of the tumour (that is to say when the tumour reached 60 mm³), treatment was started with Abo1 and pemetrexed administered as follows: pemetrexed at a dose of 100 mg/Kg in 88 ml/mouse for 5 consecutive days intraperitoneally, artichoke extract in drinking water at concentrations of 25, 50 and 75 micrograms/ml and measured on alternate days for a period of 3 weeks.

The mice were monitored daily to evaluate any signs; body weight was monitored twice weekly.

At the end of the experiment (42 days after inoculation), the tumour masses were collected and fixed in 10% formalin (transferred after 24 hours to 70% ethanol).

The tumour diameters were measured twice weekly using a Mitutoyo caliper.

### 12. Generation of Hypoxia Model

Scientific literature of the last years describes the relevance of tumour microenvironment in the development and progression of the tumour itself. Following a scarce and abnormal vascular development, most solid tumours present oxygen levels lower than those of normal tissue, with the consequent forming of hypoxic areas which induce adaptive changes associated with a more metastatic phenotype and a greater resistance to therapies.

In this context, the hypoxia-induced transcription factor (HIF-1) mediates cell responses to stress and controls the expression of many genes involved in glycolysis regulation, glucose transportation, cell survival and proliferation, angiogenesis and metastasis.

The increase of HIF-1 activity in tumours derives from two concomitant factors: higher expression of HIF-1alpha, regulatory subunit of the protein, and constitutive activation of STAT3, lead to deregulation of the GSH/GSSG system, with entailed GSH increase causing a higher survival of tumour cells and their greater resistance to chemotherapeutic agents.

Among the various metabolic and signal transduction pathways altered in the tumour environment, two main targets were singled out in order to control hypoxia-induced adaptive events and induce cell death: HIF-1alpha and the GSH/GSSG system. The authors demonstrated that the extract of Cynara scolymus is a powerful inhibitor of STAT3, induces apoptosis and makes some tumour lines more sensible to some chemotherapeutic agents.

Some cell lines, derived from various human tumours, were cultivated under hypoxia by using a RUSKIN incubator in which a mixture of gases (O₂, CO₂ and N) with varying percents is blown.

Two hypoxia models were generated:
ACUTE HYPDXIA O₂<2% up to 48 hours
CHRONIC HYPDXIA O₂<2% for long times.

12.1 Cell Cultures

Human breast cancer cell lines T47-D and MDA-MB231, human colorectal adenocarcinoma line HT-29, human prostate cancer line DU145, human uterine cervix carcinoma cell line HeLa, human hepatocarcinoma cell line HepG2 (ATCC, American Type Culture Collection) were cultivated in DMEM (BioWhittaker, Cambrex Bio Science, Belgium) in a 5% CO₂ atmosphere at 37° C.

The culture medium was integrated with 10% bovine fetal serum (FBS, BioWhittaker, Cambrex BioScience, Belgium), 100 UI/ml of penicillin, 100 mg/ml of streptomycin, and 40 mg/ml of gentamycin.

To establish a chronic hypoxia model, the above cell lines were cultivated in DMEM with 10% FBS in 5% CO₂ and 1% O₂ atmosphere, balanced with N using a multigas incubator (Ruskinn C300, Ruskinn Technology Ltd., Bridgend, United Kingdom). Cells survived through each cell passage were selected and cultivated with reoxygenation and hypoxia cycles for at least 3 months and up to 6 months.

12.2 Cell Vitality

WST-1

Cell vitality (viability) was measured by colorimetric assay based on cleavage of tetrazolium salt 4-[3-(4-Iodophenyl)-2-(4-nitrophenyl)-2H-5-tetrazolium]-1,3-benzene disulfonate WST-1 (Roche Molecular Biochemicals Indianapolis, Ind.) to formazan by mitochondrial dehydrogenase.

TrypanBlue/Countess

Cell vitality was assessed with a 0.4% Trypan Blue exclusion test, using an automated Countess cell counter (Life Technologies™).

12.3 Proliferation Assay

CFSE proliferation assay was used to monitor distinct generations of proliferating cells by dye dilutions. CFSE is a cell membrane-permeable fluorescent molecule entering the cell and binding to amino groups on proteins, with entailed long-term retention of the dye inside the cell. Through successive cell divisions, each daughter cell receives about one half of parent's fluorescence.

Analysis of cell population fluorescence intensity by flow cytometry allows the determining of the number of generations through which a cell or a population has progressed from the labeling. Each generation of cells appears as a different peak on a flow cytometry histogram.

Cells were washed twice with PBS, centrifuged and then resuspended in 0.1% PBS/BSA, adjusting cell density to 1×10⁶. A concentration of 10 μM dye was added to the cell suspension and the whole was incubated for 10 minutes at 37° C., protected from light. After 5 minutes of incubation on ice, cells were washed three times with 0.1% PBS/BSA to remove free dye remaining in the solution, resuspended in the culture medium and distributed in 100,000 cell-aliquots to be stained in multi-well plates. After 24, 48 and 72 hours, cells were collected and analysed by FACS equipped with a 488-nm excitation source.

12.4 Cell Cycle Analysis

Cell cycle analysis was obtained by performing univariate analysis of deoxyribonucleic acid (DNA) content.

Cells were harvested at 24, 48 and 72 hours and stained with the (cell-permeable) fluorescent dye Vibrant Orange, which, after binding with the double-strand DNA, emits a fluorescent signal proportional to the DNA mass. Flow cytometry was used to measure fluorescence intensity of individual cells. By using this approach, a frequency distribution histogram or DNA frequency histogram can be made in order to show cell cycle phases G1/G0, S and G2/M.

12.5 Quantification of L-Lactate, Piruvate and Glucose

L-lactate, piruvate and glucose were quantitated in the supernatants both of hypoxic and normoxyc cells by spectrophotometer after having carried out specific enzymatic reactions, according to L-lactic acid Assay (Meganzyme) and Glucose Colorimetric Assay (Cayman) procedures

12.6 Western Blotting Analysis

Cells were homogenized at 4° C. in 20 mM HEPES, pH 7.4, containing 420 mM NaCl, 1 mM EDTA, 1 mM EGTA, 1% Nonidet-P40 (NP-40), 20% glycerol, protease inhibitor cocktail (GE Healthcare, Amersham Place, United Kingdom) and phosphatase inhibitor cocktail. Aliquots of cell lysate were loaded (40 μg of total proteins per lane) on 7.5% SDS-polyacrylamide gel. Electrophoresis was carried out at 100V with a run buffer containing 0.25 M Tris HCl, pH 8.3, 1.92 M glycine, and 1% SDS. Resolved proteins were subjected to electroblotting on a PVDF membrane (Immobilon P, Millipore, Bedford, Mass.) and incubated with appropriate antibodies overnight at 4° C. After rinsing, the membranes were developed with peroxidase-conjugated anti-rabbit or anti-mouse IgG antibodies (Cell Signaling Technology) and a chemiluminescence detection system (Kit Immun-Star™ WesternC™ Bio-Rad, Hercules, Calif.). Proteins subjected to blotting were detected and quantitated by using ChemiDoc XRS Imaging System (Bio-Rad).

12.7 Acute Hypoxia

The cell lines indicated in the Table below were cultivated for 24 or 48 hours in a Ruskin Cabinet at 37° C., under an atmosphere with three different O₂ concentrations (0.5%, 1% and 2%) and cell vitality was analysed both with WST-1 colorimetric assay, based on the reduction of tetrazolium salts by mitochondrial dehydrogenase present in live cells, and with Trypan blue associated to the count by COUNTESS/INVITROGEN.

| Cell line | |
|---|---|
| T-47D | Human breast cancer |
| MDA-MB231 | Human breast cancer |
| HeLa | Human cervical tumor |
| HepG2 | Human liver cancer |
| DU-145 | Human prostate cancer |
| HT-29 | Human colon cancer |

12.8 Chronic Hypoxia

In order to re-create a state of chronic hypoxia, present in the interior of a solid tumour, all cell lines indicated in the table above were cultivated for some months in the Ruskin Cabinet at 37° C., under a 1% O₂ atmosphere. The medium was changed every three days so as to eliminate dead cells, and approximately every 10-15 days the cells were placed in the incubator under normoxia conditions for 24 hours.

Among all cells analysed, only MDA-MB231 cell line adapted to the hypoxic environment and survived. MDA-MB231 cell line, adapted to chronic hypoxia, was denominated ChR-MDA-MB231 (chronic hypoxia-resistant).

These cells have slow growth, and after some months exhibit a completely different morphology. To better highlight this change, cells were treated with the Cell-mask dye specific for cell membranes and photographed with a confocal microscope. ChR-MDA-MB231 cells have a more fusiform shape compared to parental cells.

Metabolism and cell proliferation were analysed. The analysis of ChR-MDA-MB231 energetic metabolism demonstrates that these cells do not consume glucose and produce scarce piruvate compared to cells cultivated under normoxia, whereas the concentration of lactate produced remains unvaried. These results demonstrated that chronic hypoxia cells ChR-MDA-MB231 have a largely slowed-down metabolism. ChR-MDA-MB231 cells were then treated with CFSE, a fluorophore that at each cell division distributes into daughter cells halving its fluorescence, thereby allowing to monitor the number of cell divisions. The results obtained show that the cells proliferate very slowly. Lastly, the cytofluorometric study of the cell cycle demonstrate that these cells are stationary in the G0/G1 phase of the cell cycle. These cells moreover do not die by anoikis but form tumourspheres, a feature typical of CANCER STEM CELLS.

To sum up, the chronic hypoxia cell model reported here presents the typical features of dormant cells that in vivo survive in the tumour for a long time, elude therapy and, following an environmental change, can give rise to metastases and recurrences. In some works they are also identified as cancer stem cells.

### 13. Tumour Cell Sensitising to Chemotherapeutic Agents Induced by the Titrated Extract of the Invention Under Hypoxic Conditions

The model created appears adapted for the study of plant extracts to be used in association with the classic chemotherapeutic agents. Then cells were treated with doxorubicin, with plant extracts or with their association, and cell vitality was analysed by Trypan Blue/Countess Invitrogen as described in the annexed Materials and Methods. The following treatments were performed:

13.1 Treatment with Doxorubicin

MDA-MB231 cells cultivated under normoxia and under chronic hypoxia were treated for 24 hours with various concentrations of doxorubicin, elective drug in the treatment of breast cancer. The data obtained demonstrate that ChR-MDA-MB231 cells are more resistant to the drug compared to parental ones cultivated under normoxia.

MDA-MB231 EC₅₀=0.25 μg/mL and chMDAMB231 EC₅₀=1 μg/mL (FIG. 29).

13.2 Treatment with the Titrated Extract According to the Invention

MDA-MB231 cells cultivated under normoxia and under chronic hypoxia were treated for 24 hours with various concentrations of the titrated extract according to the invention, dissolved in 50% EtOH at the concentration of 100 mg/ml. The results obtained demonstrate that the extract used inhibits cell vitality in a dose-dependent manner (FIG. 30) (EC₅₀=300 ug/mL).

13.3 Treatment with the Titrated Extract According to the Invention and Doxorubicin

In order to sensitise cells to chemotherapeutic treatments, chMDA-MB231 cells were treated with increasing doses of the titrated extract according to the invention, in combination with 0.25 ug/ml of doxorubicin as indicated in FIG. 31, and cell vitality was assessed with Trypan blue as indicated below. For this type of experiments, a doxorubicin amount able to induce a 20% mortality in the cells under examination was used.

The results demonstrate that the extract sensitises cells to treatment with doxorubicin.

14. Determination of Chlorogenic Acid and Cynaropicrin in Cynara scolymus

Sample preparation: weigh 0.25 g of lyophilised extract (0.5 g of ground leaves) and extract with 50 ml of 75% MeOH/0.1% HCOOH under ultrasound for 15 min, protected from light. Centrifuge and decant in a 100 ml volumetric flask. Repeat on the residue a second extraction under the same conditions. Centrifuge and decant in the same 100 ml flask. Bring to volume the reunited organic extracts at 20° C. with the same extraction solvent. Filter over a 0.45 μm cellulose acetate filter and inject into a UHPLC or HPLC system.

Chromatographic conditions (UHPLC):

Column: Poroshell 120 EC-C18, 3×100 mm 2.7 μm+In-line filter 4.6 mm, 0.2 μm

filter; column temperature: 30° C.±0.8° C.

detector: Diode Array Detector

CHLOROGENIC ACID: wavelength=325 nm bandwidth 4.

CYNAROPICRIN: wavelength=212 nm bandwidth 4.

flow rate: 0.43 ml/min.

injection volume: 5 μl

mobile phase: A=H2O/0.1% HCOOH, B=CH3CN/0.1% HCOOH.

Elution conditions:

| min. | A % | B % |
|---|---|---|
| 0 | 92 | 8 |
| 15 | 52 | 48 |

stop time: 15 min.
post time: 5 min.

Standard preparation:

Standard: Cynaropicrin Solubilization solvent: MeOH for HPLC. Working concentration: from 0.00404 to 0.064624 mg/ml. Storage conditions: working solutions are stored at 20° C. and protected from light.

Standard: Chlorogenic acid solubilization solvent: 50% MeOH for HPLC. Working concentration: 0.02548 to 0.10192 mg/ml. Storage conditions: working solutions are stored at +4° C. and protected from light.

Chromatographic Conditions (HPLC Method):

Column: Luna C18 150×4.6 mm 5 μm

column temperature: 30° C.±0.8° C.

detector: Diode Array Detector

CHLOROGENIC ACID: wavelength=325 nm bandwidth 4. Ref. off

CYNAROPICRIN: wavelength=212 nm bandwidth 4. Ref.off

flow rate: 0.5 ml/min.

injection volume: 10 μl

mobile phase: A=H2O/0.1% HCOOH, B=CH3CN/0.1% HCOOH

Elution conditions:

| min. | A % | B % |
|---|---|---|
| 0 | 92 | 8 |
| 38 | 62 | 38 |
| 45 | 5 | 95 |

stop time: 45 min.
post time: 10 min.

Calculations:

The percent content of Chlorogenic acid in solid products is calculated with the following formula:

$$\begin{matrix}\% Chlorogenic & \\ acid in solid & \frac{A C \times conc . St \times V}{ASt \times p \times 10} \\ products = & \end{matrix}$$

Where:

AC=area of peak related to chlorogenic acid in the sample;

Ast=area of peak related to chlorogenic acid in the standard;

conc.st=conc. in mg/ml of chlorogenic acid in the standard;

V=total volume in ml of the extract;

p=sample weight in grams;

F=dilution factor.

The percent content of cynaropicrin in solid products is calculated with the same formula.

15. Determination of Total Caffeoylquinic Acids Expressed as Chlorogenic Acid in Cynara scolymus.

Preparation of sample: accurately weigh 0.30 g±0.015 g of lyophilised extract sample (0.50 g if ground leaves). Add 40 ml of ultrapure H₂O and place under magnetic stirring at the temperature of 95° C.±2° C. Upon reaching the boiling temperature, filter through cotton in a 50 ml centrifuge tube. Add 2 ml of a saturated acetate lead solution to the (still warm) solution.

Cool down, centrifuge and discard the supernatant. Add 5 ml of ultrapure H₂O to the residue and stir the centrifuge tube. Centrifuge again and discard the supernatant. Extract the residue with 70 ml of diluted acetic acid (11.4 ml brought to 100 ml with ultrapure H₂O) and heat to boiling under slow stirring. Filter through cotton the still warm solution and add 2 ml of a (200 ml/I) solution of sulphuric acid. Centrifuge and decant the clear solution in a 100 ml volumetric flask. Add 5 ml of diluted acetic acid to the residue. Centrifuge and decant the clear solution in the same 100 ml flask. At room temperature, bring to a 100 ml volume with diluted acetic acid.

Test solution: take 1 ml of solution. By volumetric flask, bring to 25 ml with methanol and stir.

Reference solution: take 1 ml of acetic acid. By volumetric flask, bring to 25 ml with methanol and stir.

Spectrophotometric Reading:

Measure test solution absorbance at 325 nm using reference solution as blank.

Definition of A1%, 1 (as defined in the European Pharmacopoeia Ed 8.0, 2.2.25)=specific absorbance, measured at a specific wavelength, of a reference substance dissolved at the concentration of 10 g/Litre, placed in a 1 cm-long cell.

Assuming the value A1%, 0.1 cm of the chlorogenic acid at 325 nm to be 485, the percent of caffeoylquinic acids, expressed as chlorogenic acid, is calculated with the formula:

Calculations:

$$\begin{matrix}\% total caffeoylquinic acids , & \\ expressed as chlorogenic & \frac{A \times Ve \times Vf}{P \times Vp \times A 1 \% , 1 cm} \\ acid = & \end{matrix}$$

wherein:

A=sample absorbance at 325 nm.

Ve=end volume of the extract.

Vf=end volume of the dilution.

p=sample weight in grams.

Vp=sample volume taken for final dilution.

A1%, 1 cm=485 (A1%, 1 cm of Chlorogenic acid, at a 325 nm wavelength).

### BIBLIOGRAPHY

Aggarwal B. B. et al. Targeting signal-transducer-and-activator-of-transcription-3 for prevention and therapy of cancer: modern target but ancient solution. Ann. N.Y. Acad. Sci. 1091; 151-69: 2006.—Berisha J. et al. Stat3 is tyrosine-phosphorylated through the interleukin-6/glycoprotein 130/Janus kinase pathway in breast cancer. Breast Cancer Research 2007, 9:R32.—Johnston P A e Grandis R G, STAT3 signaling: anticancer strategies and challenges. Mollnterv; 11 (1); 18-26:2011.
Niu G. et al. Signal transducer and activator of transcription 3 is required for hypoxia-inducible factor-1alpha RNA expression in both tumor cells and tumor-associated myeloid cells. Mol Cancer Res, 6 (7); 1099-105: 2008.
Tsao et al. Inhibition of c-Src expression and activation in malignant pleural mesothelioma tissues leads to apoptosis, cell cycle arrest, and decreased migration and invasion. Mol Cancer Ther 2007; 6:1962-1972.
Turkson J. Jove R. “STAT proteins: novel molecular targets for cancer drug discovery” Oncogene. 2000 Dec. 27; 19(56):6613-26.
Yu. H. et al. “STATs in cancer inflammation and immunity: a leading role for STAT3” Nature Reviews Cancer 9, 798-809 (November 2009).

1. An extract of Cynara scolymus or a fraction of extract of Cynara scolymus or a mixture of said extract with one or more of said fractions or a mixture of said fractions, titrated in total caffeoylquinic acids, in chlorogenic acid and in cynaropicrin, wherein
total caffeoylquinic acids represent from 8% to 16% by weight of said extract or of said fraction or of said mixture in dry form, chlorogenic acid represents from 3.5% to 7% by weight of said extract or of said fraction or of said mixture in dry form, and said cynaropicrin represents from 0.2% to 4% by weight of said extract or of said fraction or of said mixture in dry form
or wherein
total caffeoylquinic acids represent from 25% to 48% by weight of said fraction in dry form, chlorogenic acid represents from 11% to 21% by weight of said fraction in dry form, and said cynaropicrin represents from 1% to 10% by weight of said fraction in dry form
in association with one or more anti-tumour or anti-inflammatory compounds.

2. The extract of Cynara scolymus or fraction of extract of Cynara scolymus or mixture of said extract with one or more of said fractions or mixture of said fractions according to claim 1, wherein
said total caffeoylquinic acids represent from 9% to 15% by weight of said extract or of said fraction or of said mixture in dry form, said chlorogenic acid represents from 3.5% to 5.5% by weight of said extract or of said fraction or of said mixture in dry form, and said cynaropicrin represents from 0.2% to 3% by weight of said extract or of said fraction or of said mixture in dry form
or wherein
said total caffeoylquinic acids represent from 25% to 35% by weight of said fraction in dry form, said chlorogenic acid represents from 11% to 15% by weight of said fraction in dry form, and said cynaropicrin represents from 1% to 8% by weight of said fraction in dry form.

3. The extract of Cynara scolymus or fraction of extract of Cynara scolymus or mixture of said extract with one or more of said fractions or mixture of said fractions according to claim 1, wherein said extract, said fraction, or said mixture is in a dry, lyophilised or fluid form and is obtained from Cynara leaves, flower-heads, or mixtures thereof.

4. The extract of Cynara scolymus or fraction of extract of Cynara scolymus or mixture of said extract with one or more of said fractions or mixture of said fractions in association with one or more anti-tumour compounds and/or anti-inflammatory compounds according to claim 1, wherein said association is carried out by concomitant or sequential administration of said extract or of said fraction, or of said mixture, with said one or more anti-tumour compounds and/or with said one or more anti-inflammatory compounds.

5. The extract of Cynara scolymus or fraction of extract of Cynara scolymus or mixture of said extract with one or more of said fractions or mixture of said fractions in association with one or more anti-tumour compounds according to claim 1, wherein said one or more anti-tumour compounds are selected from the group consisting of cisplatinum, doxorubicin, pemetrexed, methotrexate, vinorelbine, gemcitabine, and taxol.

6. A method of using the extract of Cynara scolymus or fraction of extract of Cynara scolymus or mixture of said extract with one or more of said fractions or mixture of said fractions in association with one or more anti-tumour compounds according to claim 1 for prevention and/or treatment of a pathological condition characterised by constitutive or anomalous activation of STAT3 transcription factor, wherein said pathological state is a tumour pathological state selected from the group consisting of: prostate cancer, multiple myeloma, leukaemia, lymphoma, melanoma, carcinoma of the ovaries, breast cancer, renal cell carcinoma, pancreatic adenocarcinoma, lung cancer, brain cancer, erythroleukemia, squamous-cell carcinoma of the head and neck, colon cancer, and malignant pleural mesothelioma.

7. The method according to claim 6, wherein said brain tumour is glioma, brain meningioma, or medulloblastoma; wherein said lymphoma is Sezary syndrome, EBV associated Buckitt lymphoma, Samiri HSV-dependent lymphoma, or cutaneous T-cell related lymphoma; wherein said leukaemia is HTLV-I-dependent leukaemia, chronic lymphocytic leukaemia (CLL), acute myelogenous leukaemia (AML), megakaryocytic leukaemia, or large granular lymphocytes leukaemia (LGL).

8. A method of using the extract of Cynara scolymus or fraction of extract of Cynara scolymus or mixture of said extract with one or more of said fractions or mixture of said fractions in association with one or more anti-tumour compounds according to claim 1 for prevention and/or treatment of a pathological condition characterised by constitutive or anomalous activation of STAT3 transcription factor, wherein said pathological condition is a tumour resistant to treatment with chemotherapeutic agents which do not inhibit STAT3.

9. A method of using the extract of Cynara scolymus or fraction of extract of Cynara scolymus or mixture of said extract with one or more of said fractions or mixture of said fractions in association with one or more anti-inflammatory compounds according to claim 1 for prevention and/or treatment of a pathological condition characterised by constitutive or anomalous activation of STAT3 transcription factor, wherein said inflammatory condition is an inflammation caused by viral infections such as infection by H. pylori, infections by hepatitis B virus, infections by HPV (human papilloma virus), or Epstein-Barr virus infections.

10. A composition comprising as active pharmaceutical ingredients:
a) an extract of Cynara scolymus or a fraction of extract of Cynara scolymus or a mixture of said extract with one or more of said fractions or a mixture of said fractions, titrated in total caffeoylquinic acids, in chlorogenic acid and in cynaropicrin, wherein
total caffeoylquinic acids represent from 8% to 16% by weight of said extract or of said fraction or of said mixture in dry form, chlorogenic acid represents from 3.5% to 7% by weight of said extract or of said fraction or of said mixture in dry form, and said cynaropicrin represents from 0.2% to 4% by weight of said extract or of said fraction or of said mixture in dry form;
or wherein
total caffeoylquinic acids represent from 25% to 48% by weight of said fraction in dry form, chlorogenic acid represents from 11% to 21% by weight of said fraction in dry form, and said cynaropicrin represents from 1% to 10% by weight of said fraction in dry form;
b) one or more anti-tumour and/or anti-inflammatory compounds;
and a carrier and/or diluent and/or excipient.

11. The composition according to claim 10, wherein
said total caffeoylquinic acids represent from 9% to 15% by weight of said extract or of said fraction or of said mixture in dry form, said chlorogenic acid represents from 3.5% to 5.5% by weight of said extract or of said fraction or of said mixture in dry form, and said cynaropicrin represents from 0.2% to 3% by weight of said extract or of said fraction or of said mixture in dry form;
or wherein
said total caffeoylquinic acids represent from 25% to 35% by weight of said fraction in dry form, said chlorogenic acid represents from 11% to 15% by weight of said fraction in dry form, and said cynaropicrin represents from 1% to 8% by weight of said fraction in dry form.

12. The composition according to claim 10, wherein said one or more anti-tumour compounds are selected from the group consisting of cisplatinum, doxorubicin, pemetrexed, methotrexate, vinorelbine, gemcitabine, and taxol.

13. A method of using the composition according to claim 10 for prevention and/or treatment of a pathological condition characterised by constitutive or anomalous activation of STAT3 transcription factor, wherein said pathological condition is a tumour pathological condition selected from the group consisting of: prostate cancer, multiple myeloma, leukaemia, lymphoma, melanoma, carcinoma of the ovaries, breast cancer, renal cell carcinoma, pancreatic adenocarcinoma, lung cancer, brain cancer, erythroleukemia, squamous-cell carcinoma of the head and neck, colon cancer, and malignant pleural mesothelioma.

14. The method according to claim 13, wherein said brain tumour is glioma, brain meningioma, or medulloblastoma; wherein said lymphoma is Sezary syndrome, EBV associated Buckitt lymphoma, Samiri HSV-dependent lymphoma, or cutaneous T-cell related lymphoma; wherein said leukaemia is HTLV-I-dependent leukaemia, chronic lymphocytic leukaemia (CLL), acute myelogenous leukaemia (AML), megakaryocytic leukaemia, or large granular lymphocytes leukaemia (LGL).

15. A method of using the composition according to claim 10 for prevention and/or treatment of a pathological condition characterised by constitutive or anomalous activation of STAT3 transcription factor, wherein said pathological condition is a tumour resistant to treatment with chemotherapeutic agents that do not inhibit STAT3.

16. A method of using the composition according to claim 10 for prevention and/or treatment of an inflammatory and/or pre-tumour condition characterised by constitutive or anomalous activation of STAT3 transcription factor, wherein said inflammatory condition and/or pre-tumour condition is an inflammation caused by viral infections such as infection by H. pylori, infection by hepatitis B virus, infections by HPV (human papilloma virus), or Epstein-Barr virus infections.

17. A kit for concomitant or sequential administration of an extract of Cynara scolymus or a fraction of extract of Cynara scolymus or a mixture of said extract with one or more of said fractions or a mixture of said fractions and one or more anti-tumour compounds and/or one or more anti-inflammatory compounds, comprising:
one or more aliquots of an extract of Cynara scolymus or a fraction of extract of Cynara scolymus or a mixture of said extract with one or more of said fractions or a mixture of said fractions, titrated in total caffeoylquinic acids, in chlorogenic acid and in cynaropicrin, wherein total caffeoylquinic acids represent from 8% to 16% by weight of said extract or of said fraction or of said mixture in dry form, chlorogenic acid represents from 3.5% to 8% by weight of said extract or of said fraction or of said mixture in dry form, and said cynaropicrin represents from 0.2% to 4% by weight of said extract or of said fraction or of said mixture in dry form, and
one or more aliquots of one or more anti-tumour compounds and/or one or more aliquots of one or more anti-inflammatory compounds.

18. The kit according to claim 17, wherein said total caffeoylquinic acids represent from 9% to 15% by weight of said extract or of said fraction or of said mixture in dry form, said chlorogenic acid represents from 3.5% to 5.5% by weight of said extract or of said fraction or of said mixture in dry form, and said cynaropicrin represents from 0.2% to 3% by weight of said extract or of said fraction or of said mixture in dry form.

19. The kit according to claim 17, wherein said one or more anti-tumour compounds are selected from the group consisting of cisplatinum, doxorubicin, pemetrexed, methotrexate, vinorelbine, gemcitabine, and taxol.

20. A method of using the kit according to claim 17 for prevention and/or treatment of a pathology characterised by constitutive or anomalous activation of STAT3 transcription factor, wherein said pathology is a tumour pathology selected from the group consisting of: prostate cancer, multiple myeloma, leukaemia, lymphoma, melanoma, carcinoma of the ovaries, breast cancer, renal cell carcinoma, pancreatic adenocarcinoma, lung cancer, brain cancer, erythroleukemia, squamous-cell carcinoma of the head and neck, colon cancer, and malignant pleural mesothelioma.

21. The method according to claim 20, wherein said brain tumour is glioma, brain meningioma, or medulloblastoma; wherein said lymphoma is Sezary syndrome, EBV associated Buckitt lymphoma, Samiri HSV-dependent lymphoma, or cutaneous T-cell related lymphoma; wherein said leukaemia is HTLV-I-dependent leukaemia, chronic lymphocytic leukaemia (CLL), acute myelogenous leukaemia (AML), megakaryocytic leukaemia, or large granular lymphocytes leukaemia (LGL).

22. A method of using the kit according to claim 17 for prevention and/or treatment of a pathological condition characterised by constitutive or anomalous activation of STAT3 transcription factor, wherein said pathological condition is a tumour resistant to treatment with chemotherapeutic agents that do not inhibit STAT3.

23. A method of using the kit according to claim 17 for prevention and/or treatment of an inflammatory and/or pre-tumour condition characterised by constitutive or anomalous activation of STAT3 transcription factor, wherein said inflammatory and/or pre-tumour condition may be an inflammation caused by viral infections (as noted in the literature), such as infections by H. pylori, infections by hepatitis B virus, infections by HPV (human papilloma virus), or infections by Epstein-Barr virus.
